(19) United States
(12) Patent Application Publication
(10) Pub. No.: US 2012/0308548 A1
(43) Pub. Date: Dec. 6, 2012

(54) METHIONINE GAMMA LYASE-2-AMINOBUTYRATE DEAMINASE (MEGL-2ABD) AND THERAPEUTIC USES THEREOF

(75) Inventor: Kallidaikurichi V. Venkatachalam, Fort Lauderdale, FL (US)

(73) Assignee: NOVA SOUTHEASTERN UNIVERSITY, Fort Lauderdale, FL (US)

(21) Appl. No.: 13/578,027

(22) PCT Filed: Feb. 10, 2011
(86) PCT No.: PCT/US2011/024290

§ 371 Date: Aug. 9, 2012

Related U.S. Application Data
(60) Provisional application No. 61/303,285, filed on Feb. 10, 2010.
(60) Provisional application No. 61/303,561, filed on Feb. 11, 2010.

(51) Int. Cl. A61K 38/50; C12N 5/071; C12N 5/095; A61P 35/00; C12Q 1/34; C12N 5/09; B82Y 5/00

(52) U.S. Cl. 424/94.6; 435/18; 435/375; 977/773; 977/915

(57) ABSTRACT

The invention relates to a methionine gamma lyase-2-aminobutyrate deaminase (MEGL-2ABD) enzyme and provides an isotopic assay for quantitatively measuring activity of the enzyme. The MEGL-2ABD is a therapeutic target for cancer, particularly in cancer stem cells and their lineages. The invention additionally provides a protein, MEGL-2ABD (SEQ ID NO:2), compositions/formulations containing this protein, and methods/assays for using this protein in therapeutic applications allowing progress toward prevention, diagnosis, treatment, and cure of cancer.

### CROSS REFERENCE TO RELATED APPLICATIONS

This application claims the benefit of priority to U.S. Provisional Application Nos. 61/303,285 (filed 10 Feb. 2010) and 61/303,561 (filed 11 Feb. 2010) the contents each of which is hereby incorporated by reference in its entirety.

### FIELD OF THE INVENTION

The invention generally relates to methionine metabolism in cells, particularly to manipulation of methionine metabolic pathways in cells, more particularly to methionine metabolism and manipulation of methionine metabolic pathways in various forms of cancer cells, including cancer stem cells, differentiated cancer cells, and proliferating cancer cells, and most particularly to the effect of methionine gamma lyase-2-aminobutyrate deaminase (MEGL-2ABD) on cancer cell growth and cell death.

### BACKGROUND

Methionine, a sulfur amino acid, is the first amino acid that is required for many proteins, during synthesis. As an essential amino acid, methionine mainly would have to come from the diet. Although considered essential, a certain amount of methionine can be synthesized de novo from homocysteine by methionine synthase using coenzymes N₅-methyl-tetrahydrofolate and methyl-cobalamin. For example, in mammals, methionine can be re-synthesized by methionine synthase [5-methyltetrahydropteroyl-L-glutamate:L-homocysteine S-methyltransferase, (E.C. 2.1.1.13; identification number from “The Comprehensive Enzyme Information System” database) using homocysteine, a methyl group from 5-methyl-tetrahydrofolate, and the coenzyme cobalamin (Foster et al. Nature 201:39-42 1964). Methionine then gets activated into s-adenosylmethionine (SAM) by methionine adenosyltransferase (EC 2.5.1.6; identification number from “The Comprehensive Enzyme Information System” database) using adenosine triphosphate (ATP) (Guest et al. The Journal of Biochemistry 92:497-504 1964; G. L. Cantoni Journal of Biological Chemistry 204:403-416 1953). SAM is the universal methyl group donor (G. L. Cantoni Journal of Biological Chemistry 225:1033-1048 1957). Once a methyl group is removed from SAM, the resulting s-adenosylhomocysteine is converted into free homocysteine. The sulfur moiety of homocysteine condenses with serine to form cystathionine with the elimination of water (H₂O), catalyzed by pyridoxal phosphate (PLP)-dependent cystathionine β-synthase (CBS) (E.C. 4.21.22; identification number from “The Comprehensive Enzyme Information System” database) (Nakagawa et al. Biochemical and Biophysical Research Communications 32:209-214 1968). Cystathionine β-lyase (CBL) (E.C. 4.4.1.1; identification number from “The Comprehensive Enzyme Information System” database), a trans-sulfurylase using PLP cleaves cystathionine at the β-position to release cysteine and the rest of the moiety is then deaminated to form α-ketobutyrate and ammonia (Flavin et al. Journal of Biological Chemistry 239:2220-2227 1964).

Recently, it has been reported that recombinant human cystathionine β-synthase (CBS) catalyzes the formation of thioether compounds lanthionine, homolanthionine and releases hydrogen sulfide (H₂S) from cysteine and homocysteine respectively (Chiku et al. Journal of Biological Chemistry 284:11601-11612 2009). In addition, H₂₅ has been reported to function as a neuromodulator in triggering the N-methyl-D-aspartate receptor and the long-term potentiation of the hippocampus, the associated memory and learning (Kimura H. Molecular Neurobiology 26:13-19 2002).

In bacteria as part of the energy related catabolism, methionine is directly cleaved between the γ-carbon and sulfur to form volatile methanethiol, a deaminated product (α-ketobutyrate, and ammonia by methionine γ-lyase (MEGL) a PLP-dependent enzyme (EC 4.4.1.11; identification number from “The Comprehensive Enzyme Information System” database) (Kreis et al. Cancer Research 33:1862-1865 1973). Methionine γ-lyase (MEGL) and its direct cleavage of methionine are absent or can not be detected in mammals.

Methionine γ-lyase (MEGL) is also known by other names such as L-methionase; methionine lyase; methioninase; methionine dethiomethylase; L-methionine γ-lyase; L-methionine methanethiol-lyase (deaminating). This enzyme, using PLP as a coenzyme, in a first step cleaves L-methionine at the γ-carbon and sulfur of the thioether bond to form methanethiol. In a second step, L-2-amino-enebutyrate is converted into α-ketobutyrate and ammonia (Kreis et al. Cancer Research 33:1862-1865 1973).

MEGL had been purified and characterized from many bacteria (Dias et al. Applied and Environmental Biology 64:3327-3331 1998; Martinez-Cuestra et al. Applied and Environmental Biology 72:4878-4884 2006; Mamaeva et al. Acta Crystallogr Sect F Struct Biol Cryst Commun. 61 (Part 6):546-549 2005; Motoshima et al. Journal of Biochemistry 128:349-354 2000; Kreis et al. Cancer Research 33:1862-1865 1973).

In addition, the genes for MEGL were isolated and the protein had been characterized from clinically-relevant organisms such as protozoa (Tokoro et al. Journal of Biological Chemistry 278:42717-42727 2003; Sato et al. FEBS Journal 275:548-560 2008; McKie et al. Journal of Biological Chemistry 273:5549-5556 1998; Coombs et al. Antimicrobial Agents and Chemotherapy 45:1743-1745 2001; Lockwood et al. Journal of Biological Chemistry 279:675-682 1991)

The MEGL protein has been implicated in oral pathology (International Publication WO 2004/055202; Yoshimura et al. Biochem. Biophys. Res. Commun, 292:964-968 2002) and identified as having an anti-tumor activity (International Publication WO 1994/11535). This anti-tumor activity makes the MEGL protein an exciting prospect for cancer therapy.

The long term objective of any cancer therapy is to find a way to destroy cancer cells and prevent relapse of the cancer. Conventional cancer cell-targeted drugs, i.e. small molecules, are not fully effective because cancer stem cells are able to expel the drugs before the cancer is destroyed and the cancer cells are then able to renew and produce relapse of the disease.

Interference with metabolic processes in cancer cells has long been a strategy in cancer therapeutics. In this regard, cellular metabolism of methionine is an optimal target.

Methionine is a sulfur-containing essential amino acid primarily obtained from the diet. In the cell, methionine levels are extremely crucial for the following metabolisms: (1) It is the first amino acid that is incorporated into many proteins during synthesis. (2) It is the precursor for cysteine synthesis through a cystathionine intermediate. (3) Methionine-derived cysteine is part of the tripeptide glutathione (γ-Glu-Cys-Gly) i.e., an endogenous antioxidant in cells (4) It is activated into s-adenosylmethionine (SAM) which is the universal methyl group donor for protein (e.g., histone) and DNA methylations, especially of CpG islands (Sen et al. Nature 463(7280):563-567 2010; Geiman et al. Mol. Reprod. Dev. 77(2):105-113 2010; Kim et al. Cell Mol. Life. Sci. 4:596-612 2009; Zheng et al. Med. Res. Rev. 5:645-687 2008; Calvisi et al. Int. J. Cancer 121(11):2410-2420; Ramani et al. Hepatology 51(3):986-995 2010). In addition to their importance in normal cells, these four pathways are relevant in cancer cell biology and have potentials in cancer therapeutics.

The MEGL-2ABD protein can be specifically targeted to cancer cells, including cancer cells of solid tumors, via incorporation into nanoparticles coated with tumor cell-specific ligands. The MEGL-2ABD cleaves methionine into α-ketobutyrate, ammonia, and hydrogen sulfide. The amino acid methionine is essential for protein synthesis of both normal and cancer cells. Decreasing the intracellular pool of methionine would knock out protein synthesis and in turn, decrease tumor survival and progression. This has great therapeutic potential since the MEGL-2ABD is absent in mammals, the therapy would have minimal side effects on normal cells when specifically delivered to cancer cells. Additionally, the side products of the MEGL-2ABD reaction, ammonia and methanethiol, are very toxic further hampering tumor cell survival.

Compared with various small molecules that are currently available to destroy cancer cells, MEGL-2ABD has advantages. Whereas small molecules are pumped out of the cell by specific ATP-binding cassettes (ABSc), MEGL-2ABD as a protein does not get pumped out from the cellular milieu and thus, hampers the overall methionine metabolism of cancer cells.

With the global study of methionine metabolism on three ontogenically and chronologically different cancer cells (stem, differentiated, and proliferating) there are potential outcomes of new diagnostics, treatment and prevention methodologies. The basic research discoveries generated in the laboratory can be translated into clinical research involving human-derived tissue. This translational research project will address drug development, biomarker development for gauging disease progression and treatment efficacy, diagnostics, and cellular therapies.

### SUMMARY OF THE INVENTION

The invention provides an alternative and promising strategy for eradicating cancer cells based upon the concept that alterations in methionine metabolism will hamper proliferation, differentiation, and renewal of cancer stem cells. The methionine gamma lysase-2-aminobutyrate deaminase (MEGL-2ABD) is a protein that, once inside the cancer stem cell, can not be pumped out like conventional drugs, and thus remains intracellular, altering methionine metabolism hampering growth of the cancer cells and spread of the cancer.

A thorough understanding of methionine metabolism in cancer cells is essential to devising cancer-targeted therapy based on MEGL-2ABD. Methionine synthase activity varies between cancer cell lines and in part this factor decides the rate of cancer cell growth and progression. The activated form of methionine is s-adenosylmethionine (SAM) that serves as the universal methyl group donor. Any alterations in SAM levels due to methionine depletion would result in defective methylation and gene expression. This epigenetic factor of methylation would alter normal cell gene expression and could result in cancer.

A thorough characterization of the MEGL-2ABD enzyme is also necessary for its successful utilization in cancer therapeutics. Conventionally, this enzyme has been characterized by measuring the formation of methanethiol by indirect DTNB spectrophotometric method. Since methanethiol is highly volatile, the kinetic parameters measured using this method need to be taken with caution. The enzyme has also been characterized by formation of α-ketobutyrate determined by a series of steps that involved chemical derivitization.

The instant invention provides a new method for characterization. In this method, the purified enzyme is tested for overall MEGL-2ABD activity by monitoring the reaction and quantifying the final product C¹⁴ α-ketobutyrate using the substrate 1-C¹⁴ L methionine. Since it measures the overall non-volatile product α-ketobutyrate, this isotope assay is very accurate.

The characterized protein was tested for anti-tumor properties. The experiments described herein show the effect of cloned and cancer cell-expressed bacterial MEGL-2ABD on cell death. Hela cells transfected with a MEGL-2ABD construct exhibited ˜30% cell death within 18 hours of transfection as judged by confocal images. Propargylglycine, an inhibitor of MEGL-2ABD, restored cells from death when included in the culture medium. Furthermore, the instant application shows the effect of MEGL-2ABD on BHK-21 and HEK-AD293T cell lines. Both BHK-21 and HEK-AD293T cell lines exhibited severe cell aggregation after 18 hours of transfection, and the cell aggregation was prevented by inclusion of propargylglycine. MTT assay on cell death supports the conclusion of confocal images which validate the effect of MEGL-2ABD on cell death, perhaps by depletion of the endogenous methionine pools.

The invention provides a protein, methionine gamma lysase-2-aminobutyrate deaminase (MEGL-2ABD) (SEQ ID NO:2), compositions/formulations containing this protein, and methods/assays for using this protein in therapeutic applications allowing progress toward prevention, diagnosis, treatment, and cure of cancer. This protein, MEGL-2ABD (SEQ ID NO:2), can be used to reduce intracellular methionine (in a cell); to increase intracellular toxicity (in a cell); and to induce cell death. These methods are particularly applicable to cancer cells including cancer stem cells, differentiated cancer cells, and proliferating cancer cells. The MEGL-2ABD protein can also be used therapeutically for eliminating and/or ameliorating cancer in a subject and is particularly applicable to targeted therapy.

Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

A more complete understanding of the present invention may be obtained by references to the accompanying drawings, when considered in conjunction with the subsequent detailed description. The embodiments illustrated in the drawings are intended only to exemplify the invention and should not be construed as limiting the invention to the illustrated embodiments.

FIG. 1 shows a schematic representation of sulfur amino acid metabolism in organisms.

FIG. 2 shows structural formulas of sulfur amino acids.

FIG. 3 shows a schematic representation of methionine resynthesis from homocysteine.

FIG. 4 shows a schematic representation of de novo cysteine synthesis.

FIG. 5 shows a schematic representation of taurine synthesis.

FIG. 6 shows a schematic representation of glutathione synthesis.

FIG. 7 shows a schematic representation of s-adenosyl methionine (SAM) synthesis.

FIG. 8 shows a schematic representation of the proposed mechanism of MEGL-2ABD on cell survival and cell death.

FIG. 9 shows the deduced amino acid sequence of MEGL/2ABD (SEQ ID NO:2; Genbank ACC number FJ875028; protein I.D. AC094451.1).

FIGS. 10A-B show purification of MEGL/2ABD on S-200 gel filtration column and purity and Mw determination by SDS-PAGE.

FIG. 11 shows a schematic representative of the MEGL/2ABD overall reaction.

FIG. 12 shows a MEGL/2ABD reaction product analysis by TLC.

FIGS. 13A-E show effects of protein, time, temperature, and pH on MEGL/2ABD.

FIG. 14 shows the effect of varying concentrations of methionine on MEGL/2ABD activity.

FIG. 15 shows HeLa, HEK-AD293T, BHK-21 cell transfection with MEGL/2ABD constructs, MEGL constructs+propargylglycine and its effect on cell death.

FIG. 16 shows results of a MTT cell viability assay.

FIG. 17 shows a schematic representation of a proposed reaction mechanism of MEGL/2ABD of Porphyromonas gingivalis.

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described proteins, methods, and any further application of the principles of the invention as described herein, are contemplated as would normally occur to one skilled in the art to which the invention relates.

Prior to commencing research, the experiments described herein were planned as follows:

### I. Research Plan

### General Outline as Follows:

The various objectives outlined in the instant application: Measure the key enzymes of methionine metabolism in cancer stem cells (CSC's), cancer differentiated cells, and cancer proliferating cells, attempt to come up with various check measures that could control cancer cell division, differentiation, metastasis, and prevention of tumor relapse through CSC's.

Study the effect of MEGL-2ABD on prostate, oral (head and neck), and breast cancer.
Measure certain essential amino acids in various types of cancer cells. This will improve understanding of cell physiology of different types of cancer cells.
Study how MEGL-2ABD kills cancer cells, particularly cancer stem cells.
Suggest MEGL-2ABD drug delivery methods.

### Specific Outline as Follows:

Alterations in overall methionine metabolism will hamper the cancer stem cell proliferation, differentiation, and renewal.

### Specific Aims

### 1. Overall Methionine Metabolism in Various Proliferating and Cancer Stem Cells.

Study the Effect of Recombinant Transfected Methionine Gamma Lyase-2 Aminobutyrate Deaminase (MEGL-2ABD) on prostate, oral (head and neck), and breast cancer. Various cancer stem cell (CSC's) (e.g., RWPE-1, mammary epithelial stem cells) and cell lines of proliferating, differentiating cancer cells will be studied with MEGL-2ABD transfections to assess the overall methionine metabolism. The following enzyme activities will be measured at various time periods and different concentrations of methionine in cell cultures in the presence or absence of MEGL-2ABD and its inhibitors.
a) Methionine Resynthesis: Methionine synthase activity under methionine deprived and methionine rich cell culture conditions will be determined.
b) In Vivo Methylation Status: b) s-adenosyl methionine synthesis (i.e., methionine adenosyltransferase and histone and DNA methyltransferase activity) will be assessed.
c) Cellular Red/Ox Status: Glutathione synthase and reductase activity will be measured.
d) De Novo Cysteine Synthesis: Cystathionine synthase and Cystathionine γ-lyase activity will be measured.
e) Overall Protein Synthesis will be assessed.

### 2. De Novo Methionine Re-Synthesis:

Methionine dependent (PC-3) and methionine independent (DU145) prostate cancer cell lines would be investigated for the effects of MEGL-2ABD on cell growth under methionine depleted and methionine rich medium. Effects of propargylglycine on MEGL-2ABD transfected cell lines would also be investigated.

### 3. Mechanism of Cell Death in MEGL-2ABD Targeted Cells:

Study the mechanism of the Apoptotic Sequences and Cell Survival sequences affected by MEGL-2ABD transfections on various cell types described above. In addition, cellular effects of methanethiol and NH₄⁺ on pH/toxicity in cancer cells will be tested.

4. Preliminary in vivo Studies/Recommendations:

Incorporation of MEGL-2ABD for therapy using viral vector or protein nanoparticle will be explored.

### Research Design, Methods, and Materials

The following protocols, methods, and materials were used when carrying out the experiments described herein.

Radionuclides [γcarboxy-¹⁴C]Methionine and ³H-L-2-aminobutyrate will be purchased from Moravek. All laboratory compounds, solvents and reagents will be purchased from Sigma-Aldrich. Rabbit polyclonal anti-p21, anti-p27, anti-Bcl-2, anti-cytochrome c, anti-p38, anti-FAK, anti-NF-κB p65, and goat polyclonal anti-survivin, anti-phospho-PKC-δ, anti-Akt, and anti-IκBα, mouse monoclonal anti-caspase-3,2, anti-PARP, various additional antibodies and related chemicals will be purchased from BD Biosciences (San Jose, Calif.). The following items will be purchased from InVitrogen Inc. or other companies: cell signaling pathway proteins, rabbit polyclonal anti-phospho-p38 (pThr180, pTyr182), anti-phospho-FAK (pTyr397) mouse monoclonal anti-PKC-8, secondary horseradish peroxidase (HRP)-conjugated anti-rabbit, anti-goat, and anti-mouse antibodies for immunostaining, R-Phycoerythrin (R-PE)-conjugated rat anti-mouse CD49d (Integrin α₄ chain), anti-mouse CD49e (Integrin α₅ chain), anti-mouse CD51 (Integrin α [symbol] chain), anti-mouse CD61 (Integrin β₃ chain), and FITC-conjugated anti-mouse CD29 (Integrin β₁ chain). Antibodies to some of the enzymes, for example cystathionine synthase (CBS), and other methionine metabolism products will be obtained from Abnova USA, Walnut, Calif. and other companies.

### Cancer Stem Cells (CSC's)

For example, teratoma and WPE-stem cells will be purchased from ATCC (American Tissue Culture Collection). WPE-stem cells are loosely adherent and exhibit features characteristic of stem/progenitor cells present in the embryonic urogenital sinus and in adult prostatic epithelium, including p63 and ABCG2. Additional Cancer Stem Cells would be purchased from Stem Cell Technologies, Vancouver, Canada. The cell culture conditions and procedures will be followed according to the respective manufacturer's instructions.

### Cell Lysate Preparation for Enzyme Activity Assays:

Pierce IP Lysis Buffer from Thermo Scientific Inc., will be used for making soluble cell lysates for enzyme assays. The same buffer will be used for isolating proteins for immunoprecipitations, Thermo Scientific Pierce BCA and 660 nm Protein Assays, protein purification, and immunoassays (e.g., ELISA, Western Blot). An aliquot from clarified cell homogenates will be used for the following enzyme assays related to methionine metabolism.

Methionine γ-lyase and Deaminase (MEGL/2ABD) Overall Activity Assays in Transfected Cancer Cells:

The overall reaction of α-ketobutyrate formation will be performed in a total volume of 24 μl. The reaction will contain 4 μl of reaction buffer [150 mM Tris-HCl (pH 8.0)], 4 μl of 1 mM pyridoxal phosphate (PLP), 3 μl of H₂O, 6 μl of storage buffer, and 4 μl of cell lysate, mixed gently, and will be incubated at 37° C. for 5 minutes. The reaction will be started by adding 1.93×10⁵ cpm of 1-¹⁴C-L-methionine (8814 cpm/nmol) and the contents will be incubated at 37° C. for 15 minutes. The reaction will be stopped by boiling for a minute, cool on ice for 2 minutes and centrifuged at 3575 g for 7 minutes. A 2 μl aliquot will then chromatographed on a silica gel plate using 25% 20 mM phosphate buffer (pH 7.0) and 75% acetonitrile. Following chromatography, the TLC plates will be dried, and exposed to x-ray film for 36-48 hours (Eastman-Kodak Co). The respective spots of substrate and products will be cut out and radioactivity determined by liquid scintillation (Venkatachalam et al. Miami Nature Biotechnology Winter Symposium 2010; Grandi et al. American Dental Association Meeting 2009; Trubey et al. HPD Research Day Abstracts p. 21 2008).

### Enzymes of Methionine and Related Metabolism

### Methionine Synthase Activity:

Will be measured according to the procedures of Yamada et al. (Journal of Nutrition 130:1894-1900 2000). Briefly, holoenzyme activity will be determined in triplicates. Reaction mixture will contain 1 mM homocysteine (Sigma-Aldrich), 50 μM AdoMet (Sigma-Aldrich), 2 mM Ti(III) citrate, 200 μM [methyl-¹⁴C]MeH₄F (Amersham Pharmacia Biotech, Uppsala, Sweden) (1 μCi/nmol), cell lysate and 150 mM potassium phosphate buffer (pH 7.2) in a total volume of 250 μL. The enzyme reaction will be carried out under a H₂ atmosphere at 37° C. for 10 minutes and then stopped by quick chilling with 2 volumes of ice-cold water. The mixture will be passed through the Dowex 1 (Cl⁻) (Dow Chemical, Midland, Mich.) column, and ¹⁴C radioactivity in the methionine-containing fractions will be measured. For total activity measurements, assay will include all components as holoenzyme assay plus 50 μM MeCbl.

### Histone and DNA Methyltransferase Activity:

Using EpiQuik assay kit (H3-K4) (Epigentek, Brooklyn, N.Y.) histone methyltransferase/inhibition would be assayed. EpiQuik DNA Methyltransferase Activity/Inhibition Assay Kit (Fluorometric) would be used to fluorometrically measure DNMT activity and inhibition.

### Glutathione Synthase Activity (Determination of Reduced Glutathione):

Using QuantiChrom glutathione assay kit (DIGT-250) from BioAssay System, Hayward, Calif. with the modified DithioNitroBenzene (DTNB) based assay, reaction of cysteine residues of GSH product that forms yellow color will be measured at 412 nM.

### Cystathionine Beta-Synthase (CBS):

CBS activity will be performed with modifications of procedures described by Frank et al. (Arch. Biochem. Biophys. 470(1):64-72 2008). Briefly, reaction will contain 1[¹⁴C]-serine with specific activity (1000 cpm/nmol) and unlabeled homocysteine. The reaction will be performed in 10 μL of Tris Buffered Saline (TBS), pH 8.6, at 37° C. Reaction will be stopped with 0.5 μL of 98% formic acid. Products will be separated on Thin Layer Chromatography (TLC) plates developed with isoproponol:water:formic acid, 40:10:3 (v/v/v). Following chromatography, the TLC plates will be dried, and exposed to x-ray film for 36-48 hours (Eastman-Kodak Co). The respective spots of substrate and products will be cut out and radioactivity determined by liquid scintillation.

Cystathionine γ-lyase (CGL) Activity:

Will be measured according to the procedures of Flavin, Chiku and Banerjee. Briefly, the formation of cysteine from cystathionine will be determined. DTNB assay will be used to measure cysteine produced. PLP dependent CGL activity assay will be followed according to the detailed procedures of Flavin and Chiku (Flavin et al. Journal of Biological Chemistry 239:2220-2227 1964; Chiku et al. Journal of Biological Chemistry 284:11601-11612 2009).

### PLP Analysis:

Briefly, the internal PLP aldimine will be removed from Fe(III)CBS (7 μM) by incubation with hydroxylamine (5 mM) in phosphate buffer (0.1 M, pH 7.2) at 4° C. for 70 hours to generate an oxime that will then be separated by ultrafiltration. The oxime will be determined fluorimetrically at 446 nm following excitation at 353 nm in an Aminco-Bowman Series 2 luminescence spectrometer. Calibration curves will be made using known concentrations of PLP and 5 mM hydroxylamine in phosphate buffer (0.1 M, pH 7.4) (Nakagawa et al. Biochem. Biophys. Res. Commun. 32:209-214 1968; Flavin et al. Journal of Biological Chemistry 239:2220-2227 1964; Chiku et al. Journal of Biological Chemistry 284:11601-11612 2009).

### Mechanisms of Cell Death in MEGL-2ABD Targeted Cells:

Will be studied using various assays. DNA fragmentation, cell cycle distribution, Western Blot Analysis using Cell Survival and Cell Death specific antibodies. Caspase assay, ECM cell adhesion assay, flow cytometry, etc., described in detail below.

### Cell Viability Assay:

The effect of MEGL on cell viability will be determined using 3-[4,5-dimethylthiazol-2-yl]-2,5-diphenyl-tetrazolium bromide (MTT, Sigma-Aldrich). For MTT assay, cells will be suspended at a concentration of ˜4×10⁴ cells (180 μl/well). 20 μl of MTT solution (5 mg/ml) will be added for each well and will be incubated at 37° C. and 5% CO₂ for about 2-5 hours for purple color development. Formazan precipitates will be dissolved in DMSO for 30 minutes at 37° C., and the absorbance will be measured at 570 nM (Kim et al. Journal of Cellular Physiology 212:386-400 2007).

### Detection of Cell Cycle Distribution:

Following transfections with MEGL-2ABD, at various time intervals, cells will be harvested, washed twice with phosphate-buffered saline solution (PBS), and will be fixed with ice-cold 70% ethanol. Samples will be stored at −20° C. for at least 12 hours, and will be washed with 1×PBS. Intracellular nuclear DNA will be labeled with 1 ml of cold propidium iodide solution containing 0.1% Triton X-100, 0.1 mM EDTA, 0.05 mg/ml RNase A, 50 μg/ml propidium iodide in PBS. Samples will be further incubated on ice for 30 minutes in the dark. Cytometric analyses will be performed using a flow cytometer (BD Biosciences). The cell cycle profile will be analyzed using manufacturer's instructions (Kim et al. Journal of Cellular Physiology 212:386-400 2007).

### Western Blots:

Cells will be lysed with the following buffer: 50 mM Tris-HCl (pH 8.0), 150 mM NaCl, 15 mM MgCl₂, 0.5% NP40, 0.1 mM Na₃V0₄, 0.1 M NaF, and protease inhibitors cocktail (Sigma-Aldrich, MO). Lysates will be kept on ice for 20 minutes, sonicated, and then centrifuged at 14,000×g for 30 minutes. Protein concentration will be determined by Lowry protein assay method. Reducing agent and loading buffer with bromophenol blue will be added to the samples, and immunoblot analysis will be performed. Proteins will be separated by 12% SDS-PAGE, and transferred on to PVDF membranes (Amersham Biosciences). Non-specific sites will be blocked with 5% milk in PBS/0.05% NP-40 (Sigma-Aldrich, MO). The membranes will be incubated with primary antibodies 0/N at 4° C. Membranes will then be washed with PBS/0.05% NP-40 and incubated with a secondary antibody conjugated to HRP (American Qualex, Calif.). The relevant protein bands will be detected by chemiluminescence using West-Phemto Supersignal Substrate (Thermo-Fisher Scientific, PA). Quantitation of bands will be performed using Image-J software program (NIH).

### DNA Fragmentation Assay:

During apoptosis (programmed cell death) specific endonucleases are activated. These endonucleases, fragment nucleosomal DNA into ˜180-200 base pairs which will be identified by agarose gel electrophoresis. After harvesting cells by centrifugation, cell pellets will be resuspended in 500 μl of lysis buffer (10 mM Tris-HCl, pH 5.0, 20 mM EDTA, 0.5% Triton X-100) and then will be incubated on ice for 25 minutes. Cell suspensions will be centrifuged at 10,000 g for 30 minutes. Supernatant of the lysates will be extracted three times with equal volumes of equilibrated phenol solution, and twice with chloroform. After the addition of 0.1 volumes of 3 M sodium acetate and 2.5 volumes of ethanol, samples will be stored at −20° C. overnight. DNA precipitate will be collected by centrifugation at 10,000 g for 5 minutes, and dried in cool air. Pellets will be washed with ice-cold 70% ethanol three times, dissolved in Tris/EDTA buffer (10 mM Tris-HCl, pH 8.0, 1 mM EDTA) containing 30 μg/ml RNase, and incubated at 37° C. for 5 hours. DNA solutions will be separated on a 2% agarose gel at 70 V. Gels will be stained with ethidium bromide. Bands will be visualized under UV (Kim et al. Journal of Cellular Physiology 212:386-400 2007).

### Caspase Activity Assay:

Caspases are serine/aspartic acid specific proteases that play a crucial role during apoptosis in degrading DNA repair proteins and various structural and regulatory proteins. Due to apoptotic signal cytochrome C from mitochondria is released, which in turn activates Caspase-9. Caspase 9 activates caspase 3 and 6 by proteolysis. Caspase-8 also activates directly or indirectly Caspase-3 and then 4,5. The following is a brief description of the assays for key caspases. Caspase activity will be measured using colorimetric DEVD-pNA (caspase-3 substrate), LEHD-pNA (caspase-9 substrate), IETD-pNA (caspase-8 substrate) and VDVAD-pNA (Caspase-2 substrate). Briefly, cells will be washed once with PBS, resuspended in lysis buffer (20 mM HEPES, pH 7.4, 100 mM NaCl, 0.5% NP-40, and 10 mM DTT), and incubated on ice for 30 minutes. After centrifugation (10,000 g 20 minutes, 4° C.), the supernatant fractions will be collected, and immediately measured for protein concentration and caspase activity. Cell lysates (50-150 μg of proteins) will be mixed with 100 μM of each caspase substrate. The mixture will then be incubated at 37° C. for 1 hour. The products formed will be measured by spectrophotometer at excitation of 400 nm and emission wavelengths of 505 nm (Kim et al. Journal of Cellular Physiology 212:386-400 2007).

### Cell-to-Extracellular Matrix (ECM) Adhesion Assay:

For ECM assay, 24-well plates will be coated with 10 μg/ml of type I collagen, type IV collagen, and fibronectin. Non-specific binding will be blocked by 2% BSA in PBS for 3 hours at 37° C. Cells treated with methanethiol produced by MEGL-2ABD will be labeled with [³H] thymidine for 2 hours at 37° C., and will be harvested. Resuspended cells will be added to the wells (2×10⁴ cells/well), and incubated for 1 hour. Unattached cells will be removed by gentle washing. Attached cells will be harvested, and the radioactivity will be determined for adhesion index (Kim et al. Journal of Cellular Physiology 212:386-400 2007).

### Flow Cytometry:

Cells will be harvested, washed twice with PBS, and incubated in PBS containing 1% FBS for 30 minutes at 4° C. Cells will then be collected by centrifugation (1,200 rpm×5 minutes), and exposed to saturating concentrations of individual PE-conjugated anti-mouse α or β₃ integrin antibodies. For β₁ integrin labeling, PE-conjugated anti-rat IgG secondary antibody will be used. After incubation on ice for 30 minutes, cells will be washed, and the relative amounts of cell surface integrins will be determined by comparison of fluorescence emission intensities collected using FACS machine.

### Statistical Analysis:

Results will be presented as mean±standard deviation (SD). All experiments will be performed three times. Data will be analyzed for statistical significance using the Student's t-test. P values of less than 0.05 will be considered significant.

### Future Direction:

Additional experiments will be conducted to delineate the apoptotic sequences that are affected by MEGL-2ABD. Methionine-dependent (PC-3) and methionine-independent (DU145) prostate cancer cell lines will be investigated for the effect of MEGL-2ABD on cell growth under methionine-depleted and methionine-rich medium.

### II. Methionine Metabolism

The study first proposes to understand methionine metabolism in various forms of cancer cells including cancer stem cells (CSCs), differentiated cancer cells, and proliferating cancer cells. Methionine levels are extremely crucial for the following reasons:

1. It is the first amino acid that is incorporated into many proteins during synthesis.
2. It is the precursor for cysteine synthesis through cystathionine intermediate.
3. Cysteine derived from methionine is part of the tripeptide glutathione (γ-Glu-Cys-Gly) i.e., cells endogenous antioxidant.
4. Methionine is activated into s-adenosylmethionine (SAM) which is the universal methyl group donor for protein (e.g. histone) and DNA methylations).

Cells are totipotent, which means every cell in our body has the ability to regenerate eventually into a tissue type or into an organ. During embryonic development of an organism the cellular events are programmed which results in an organ and eventual morphogenesis of the embryo into an organism. In a fully developed organism stem cells that are present in various organs of the body are the embryonic reminiscent. These stem cells, (for example bone marrow cells), undergo cell division and regenerate tissues based on cellular half lives. White blood cells regenerate approximately once in a week and red blood cells regenerate once in 3-4 months. Various other organs like spleen, liver, etc., have longer half lives of regeneration. Brain, although considered plastic in its regenerative capacity, is limited in its ability to regenerate into a new tissue and compartmentalized compared to other tissues. Thus, tissue regeneration from its primordial pluripotent stem cells is an ongoing fundamental principle of life (Leeb Stem Cell Rev. 2009).

This process of cell division regulation is prone to various influences of environmental insults and cellular nutritional status. Such epigenetic influences can sometime cause mutations in the normal stem cells to become cancer stem cells popularly known as (CSC's) or tumor initiating cells (Lewis et al. Breast Cancer Research 12(1):101 2010; Charafe-Jauffret et al. Cell Cycle 9(2):229-230 2010; Nie, D. Front Bioscience (school edition) 2:184-193 2010; Rudin et al. Journal of Thoracic Oncology 11:Suppl 3:S1079-1081 2009; Reynolds et al. Cell Stem Cell 5(5):466-467 2009; Hadjipanayis et al. Trends in Molecular Medicine 11:519-530 2009; Oliveira et al. Histol. Histopathology 25(3):371-385 2010; Tang et al. International Journal of Clinical and Experimental Pathology 3(2):128-138 2009). These tumor initiating stem cells (CSC's), under certain epigenetic influences, can differentiate into more aggressive cancer cells that develop into a cell mass. This cell mass becomes metabolically self sufficient by forming new blood vessels (angiogenesis) that supply oxygen and various nutrients for survival and spreading/invading into various other regions of the body (Brown et al. Expert Rev. Hematol. 2(9):145-158 2009; Frank et al. The Journal of Clinical Investigation 120:41-50 2010; Schatton et al. Bioessays 31:1038-1049 2010; Eyler et al. Journal of Clinical Oncology 26:2839-2845 2008; Fabian et al. Cytometry Part A 75A:67-74 2009). This tenacity of cancer cells to relapse is a challenge for successful therapeutics. While considering whole body cancer therapeutics the other major challenge is to ensure that the therapeutic target/drug hampers only the cancer cell populations and not the normal cells (Brown et al. Expert Rev. Hematol. 2(9):145-158 2009; Frank et al. The Journal of Clinical Investigation 120:41-50 2010; Schatton et al. Bioessays 31:1038-1049 2010; Eyler et al. Journal of Clinical Oncology 26:2839-2845 2008; Fabian et al. Cytometry Part A 75A:67-74 2009; Park et al. Journal of Clinical Investigation 120(2):636-644 2010; Okuno et al. Oncology Report 2:485-492 2010; Mueller et al. Front Bioscience (Elite Edition) 2:602-613 2010). Metabolically and with reference to gene expression, cancer cells are quite different. For example, there are large numbers of hematological malignancies like acute myeloid leukemia (AML), B-cell acute lymphoblastic leukemia (B-ALL), multiple myeloma, T-cell acute lymphoblastic leukemia (T-ALL) express cell surface receptors CD-34. These cell surface markers can be used as a ligand to deliver cargos (i.e. drugs) that would enter only cancer cells and could destroy them. In solid tumors, for example CNS, colon, Ewing's, pancreas and prostate, they express CD44 whereas breast, head and neck, melanoma, liver, ovarian, express CD133 cell surface markers (Frank et al. The Journal of Clinical Investigation 120:41-50 2010). Thus, among various CSC's there are clear distinctions in cell surface protein expression which can be taken advantage for specific drug delivery (Frank et al. The Journal of Clinical Investigation 120:41-50 2010). Metabolically, cancer CSC's are very unique. CSC's are able to a) self renew, b) differentiate and c) proliferate and cause tumor. These three processes involve numerous metabolic activities that are unique (Telford, W. G. Current Protocols Cytom. Chapter 9:Unit 9:30 2010; Harrison et al. Cancer Research 70(2):709-718 2010). However, one process that must be common among the CSC's and its progenitors is the synthesis of new proteins. In eukaryotes, the first amino acid that is incorporated into protein is methionine. In prokaryotes, it is the formyl group modified methionine (f-Met) that is the first amino acid which is incorporated into proteins. The levels of methionine are extremely crucial for cancer cell survival. The metabolism of the sulfur amino acid methionine and its various associated pathways that can regulate the methionine pool which in turn can regulate the cell division, differentiation, and, proliferation of cancer stem cells is described herein.

### Methionine and the Related Sulfur Amino Acid Cycle:

Methionine levels are extremely crucial for the following metabolisms. 1. It is the first amino acid that is incorporated into many proteins during synthesis. 2. It is the precursor for cysteine synthesis through cystathionine intermediate. 3. Cysteine derived from methionine is part of the tripeptide glutathione (γ-Glu-Cys-Gly) i.e., cells endogenous antioxidant. 4. Methionine is activated into s-adenosylmethionine (SAM) which is the universal methyl group donor for protein (e.g., histone) and DNA methylations, especially of CpG islands (Sen et al. Nature 463(7280):563-567 2010; Geiman et al. Mol. Reprod. Dev. 77(2):105-113 2010; Kim et al. Cell Mol. Life. Sci. 4:596-612 2009; Zheng et al. Med. Res. Rev. 5:645-687 2008; Calvisi et al. Int. J. Cancer 121(11):2410-2420; Ramani et al. Hepatology 51(3):986-995 2010). These four pathways are relevant in cancer cell biology and have potentials in cancer therapeutics.

From the extracellular milieu inorganic sulfate anion is transported into intracellular regions by specific sulfate permeases. Once inside the cell, sulfate is activated into 3′-phosphoadenosine 5′-phosphosulfate (PAPS). PAPS, is the universal sulfonate donor. There are many extracellular matrix (ECM) components like proteoglycans of eukaryotes that gets sulfonated by PAPS. Upon sulfonation these macromolecules are altered in its physicochemical properties. ECM proteins are quite different between normal and cancer cells (Calvisi et al. Int. J. Cancer 121(11):2410-2420; Ramani et al. Hepatology 51(3):986-995 2010). In bacteria, PAPS is reduced into sulfide by complex sets of enzymes and sulfide is then incorporated into cysteine by de novo mechanisms. The de novo synthesis of cysteine from sulfide through reduction of PAPS is absent in mammals.

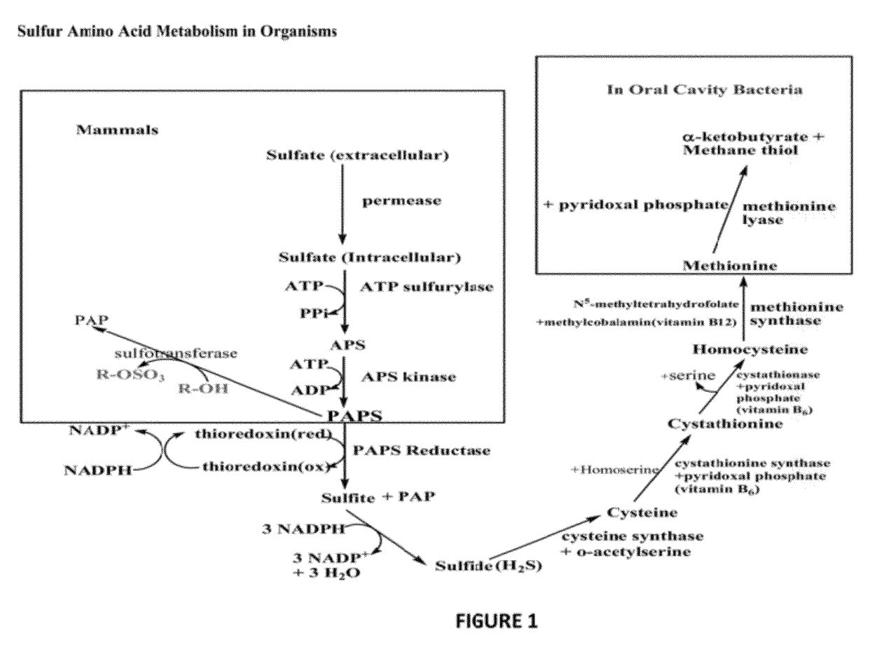

In addition, in bacteria as part of the energy related catabolism, methionine is directly cleaved between the γ-carbon and sulfur to form volatile methanethiol, a deaminated product α-ketobutyrate and ammonia by methionine γ-lyase/2-amino butyrate (MEGL-2ABD) a PLP dependent enzyme (EC 4.4.1.11; identification number from “The Comprehensive Enzyme Information System” database) which is absent in mammals (FIG. 1). The proposed relevance of MEGL-2ABD in cancer biology and its therapeutics is discussed further below (Hoffman, R. M. In Vitro 18:421-428 1982; Sun et al. Cancer Research 63:8377-8383 2003; Hoffman et al. Proceedings of the National Academy of Science USA 73:1523-1527 1976; Hori et al. Cancer Research 56:2116-2122 1996; Takakura et al. Cancer Research 66:2807-2814 2006; Kim et al. Journal of Cellular Physiology 212:386-400 2007).

### Methionine Metabolism and its Cancer Relevance:

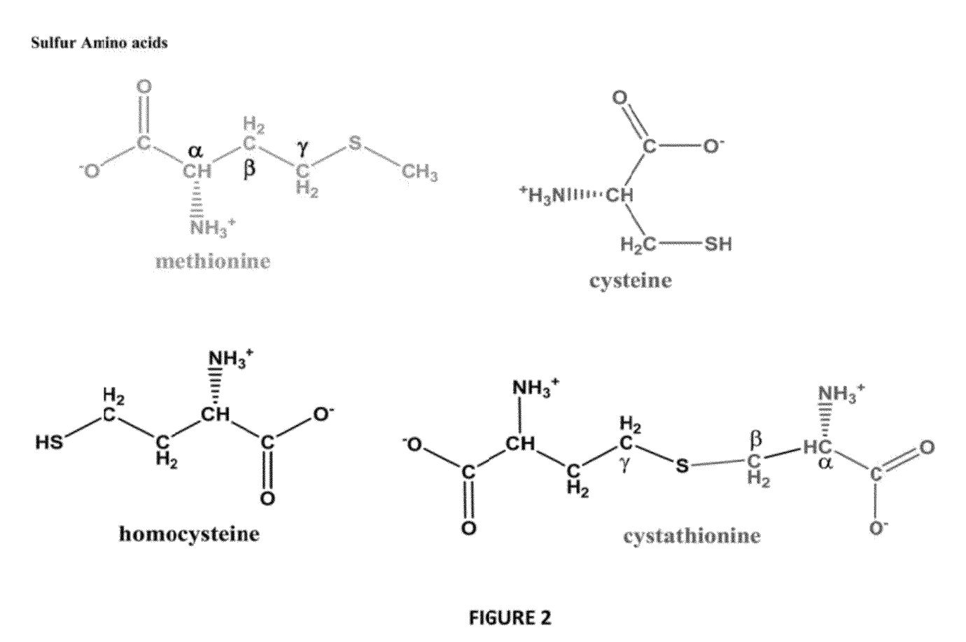

1. The physiological concentration of methionine is around 1 mM. The major portion of it is channeled for making new proteins for which, methionine is essential. The three major sulfur amino acids that control the sulfur amino acid pools are methionine, cysteine, homocysteine and cystathionine (FIG. 2).

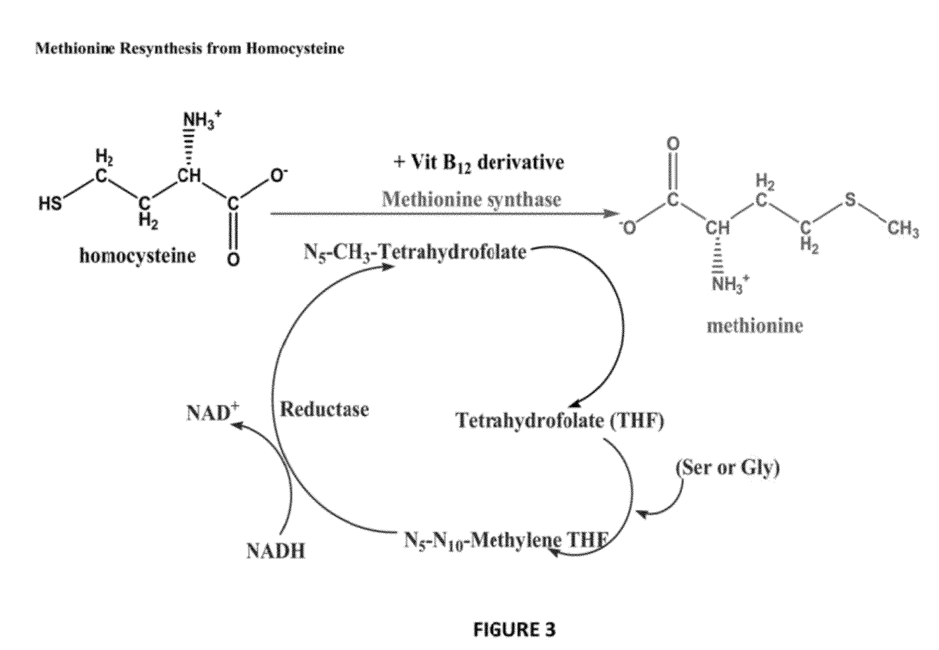

Ordinarily methionine is obtained from the diet. It is considered an essential amino acid since it is required in larger quantities during development. However, limited quantities of methionine can be synthesized endogenously by methionine synthase (FIG. 3). In this reaction, homocysteine, a non-protein amino acid, is converted into methionine by methionine synthase. Methionine synthase requires coenzymes N₅-methyl-tetrahydrofolate (a vitamin B₉ derivative) and cobalamin (a vitamin B₁₂ derivative) (Weissbach, H. Journal of Biological Chemistry 35:23497-23504 2008; Foster et al. Nature 201:39-42 1964; Guest et al. Biochemistry Journal 92:497-504 1964; Yamada et al. Journal of Nutrition 130:1894-1900 2000). This complex pathway is poorly understood metabolically, especially in cancer stem cells. Among all the metabolic steps in cells there are only two enzymes that require vitamin B₁₂ derivative as a coenzyme and the other step unrelated to this topic, is the conversion of propionyl CoA into succinyl CoA. Many nutritional studies related to folate (vitamin B₉) and cobalamin (vitamin B₁₂) during cell division and metastasis have addressed the issue of vitamin excess and its probable cancer effects.

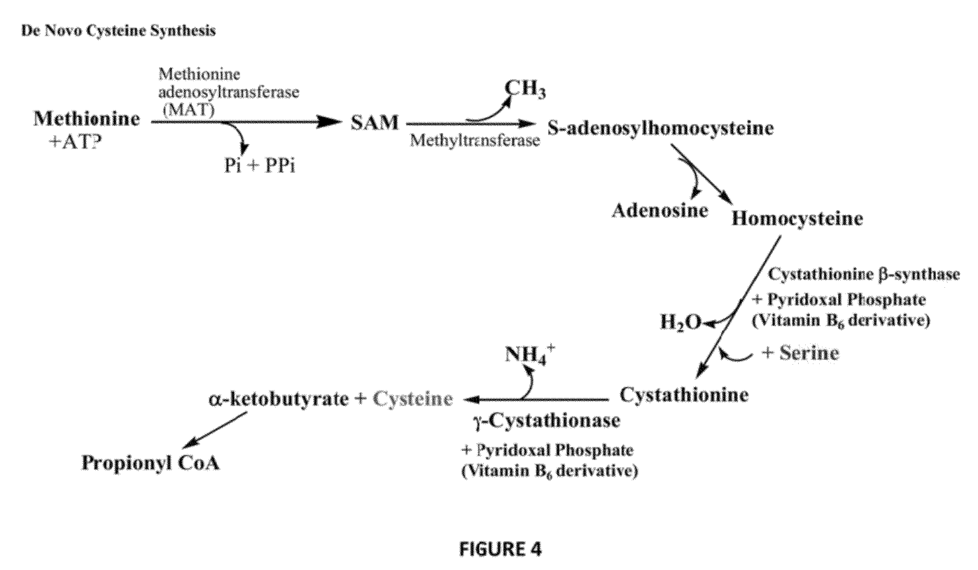

2. De Novo Cysteine Synthesis: Cysteine is considered a nonessential amino acid since it can be synthesized from methionine (FIG. 4) (Vitvitsky et al. Am. J. Physiol. Regul. Integr. Comp. Physiol. 287:R39-R46 2004; Watanabe et al. Proceedings of the National Academy of Science USA 92:1585-1589 1995; Kery et al. Archives of Biochemistry and Biophysics 355(2):222-232 1998; Nakagawa et al. Biochemical Biophysical Research Communications 32:209-214 1968; Flavin et al. Journal of Biological Chemistry 239:2220-2227 1964; Chiku et al. Journal of Biological Chemistry 284:11601-11612 2009; Frank et al. Archives of Biochemistry and Biophysics 470(1):64-72 2008). The key intermediates in this pathway are s-adenosyl methionine (SAM), the universal methyl group donor, and cystathionine. Cystathionine is cleaved at the γ position to form cysteine and the deaminated product α-ketobutyrate. Cysteine itself can be incorporated into proteins or into cells endogenous anti oxidant glutathione synthesis or converted into taurine. The α-ketobutyrate itself can be completely oxidized to provide energy for cell. Both cystathionine synthase (CBS) and γ-cystathionase requires the coenzyme pyridoxal phosphate (a vitamin B₆ derivative) (Clausen et al. Journal of Molecular Biology 262:202-224 1996; Percudani et al. EMBO Reports 4:850-854 2003; Eliot et al. Annual Review of Biochemistry 73:383-415 2004). Thus, various vitamins in sulfur amino acid metabolism are important. Therefore, in dividing CSC's or differentiated cancer cells the concentration of some of the B vitamins and sulfur amino acid pool is extremely crucial to regulate the cell cycle.

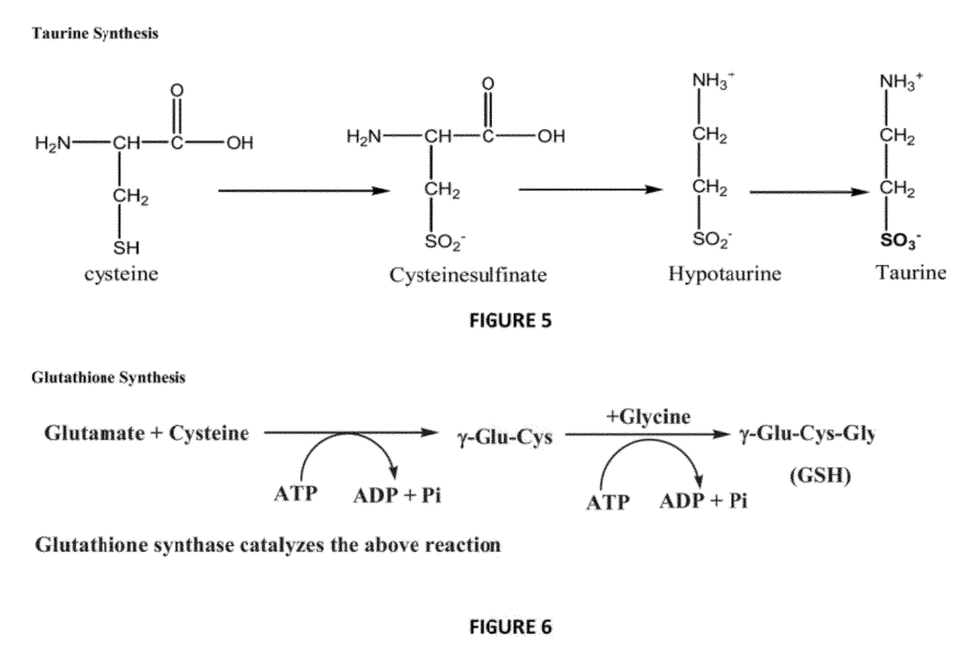

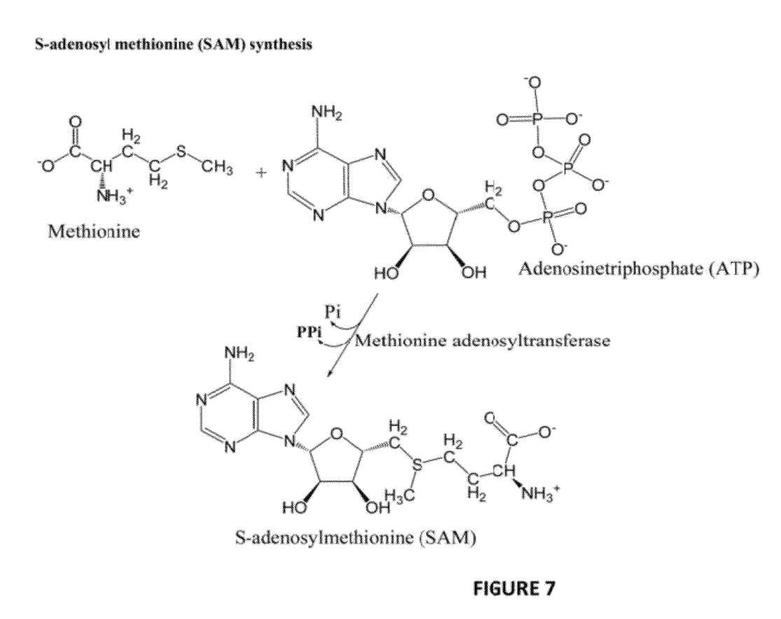

Taurine, which is considered a neurotransmitter, can be synthesized from cysteine. Eventually, taurine is catabolized into sulfate (FIG. 5). Thus, the in vivo sulfur cycle and its metabolism are crucial during cell division and cell progression.

3. Cells Antioxidant Glutathione: The levels of glutathione (γ-Glu-Cys-Gly) are indirectly controlled by methionine sulfur amino acid cycle. Glutathione is a cysteine containing tripeptide that controls cellular red/ox states. In a differentiating cancer cell there is a tremendous amount of energy utilization which in turn can result in free radical formation. Free radicals generated due to excessive metabolic activities (i.e. prolonged cell division) are inactivated by a multistep process that requires a reduced form of glutathione (GSH). The levels of GSH must be maintained as high as 5 mM for proper levels of antioxidant functions in normal cells. In cancer cells, due to uncontrolled cell division the levels of GSH are far too low to handle the physiological loads of oxidative metabolism (Lu, S.C. Molecular Aspects of Medicine 1-2:42-59 2009; Di Pietro et al. Expert Opinion on Drug Metabolism and Toxicity 2:153-170 2010; Yang et al. Biochemical Journal 391 (Part 2):399-408 2005; Yang et al. Molecular and Cellular Biology 14:5933-5946 2005).
4. S-adenosyl methionine (SAM) synthesis: S-adenosylmethionine (SAM) is the universal methyl group donor. Methylation is a major epigenetic process that modulates histone proteins that wraps up DNA in chromatin. There are specific methyl transferases that can methylate DNA and histones. Alterations in methylation can result in changes of gene expression. There are many biomolecules ranging from low to high molecular weight compounds that are modified by methylation (Cantoni. G. L. Journal of Biological Chemistry 204:403-416 1953; Cantoni et al. Journal of Biological Chemistry 225:1033-1048 1957). In cells, SAM is synthesized de novo from methionine and ATP by methionine adenosyltransferase (EC 2.5.1.6; identification number from “The Comprehensive Enzyme Information System” database) using ATP. Once a methyl group is removed from SAM, the resulting s-adenosylhomocysteine is converted into free homocysteine. Thus, we have seen the importance of methionine for the synthesis of 1. Proteins 2. Cysteine 3. Glutathione and 4. SAM. Hence, if one were to alter the endogenous levels of methionine specifically in cancer cells then one could hamper the cell division of the CSC's and eventually the differentiation and proliferation of cancer cells and could potentially prevent cancer relapse as well.

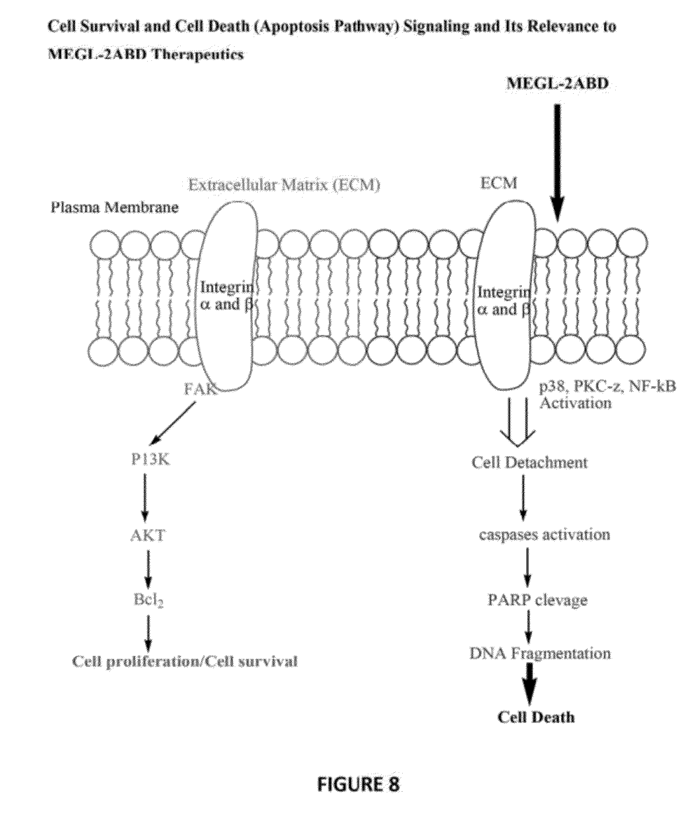

During the cell cycle, cells can take up the proliferation/survival pathway which involves key sequences of signaling proteins (Hoffman et al. Proceedings of the National Academy of Science USA 73:1523-1527 1976; Hori et al. Cancer Research 56:2116-2122 1996; Takakura et al. Cancer Research 66:2807-2814 2006; Kim et al. Journal of Cellular Physiology 212:386-400 2007). For example, Extracellular Matrix (ECM) protein gets stimulated by hormones or agents that can trigger Focal Adhesion Kinase (FAK), which in response can stimulate P13K, and in a sequential manner AKT and bcl₂ are triggered respectively. The end result is cell proliferation/cell survival. In the cell death pathway, it is hypothesized that due to the MEGL-2ABD in vivo transfections, methionine levels would be low (FIG. 8). Many different pathways are affected since methionine is the crucial amino acid and cells are starved to death. In addition, the excessive methanthiol produced would be toxic to cells and the NH₄⁺ produced would upset the cellular pH. All these factors would trigger the apoptotic pathway. The following sequences of signaling events in the apoptotic pathway would be stimulated. Low methionine would signal the activation of p38, PKδ, NF-κB. This in turn would trigger cell detachment, activation of Caspases, PARP cleavage and DNA fragmentation and eventual cell death. Thus, using antibodies to various cell survival and cell death proteins/kinases the up or down regulations of the pathway would be delineated by western blot and enzyme assays.

III. A Experimental: The Role of Methionine-γ-lyase in Oral Pathology

Oral malodor (halitosis) is caused mainly by volatile sulfur compounds (VSCs), such as hydrogen sulfide, methyl mercaptan, and dimethyl sulfide. These VSCs can either be endogenous or exogenous in origin. Exogenous VSCs are found in the diet while endogenous VSCs originate from bacterial enzymatic breakdown of certain substrates found anywhere from the stomach to the oral cavity. These compounds are highly toxic, especially methyl mercaptan, and have been shown to increase the permeability of the oral mucosa and decrease protein or collagen synthesis in the mouth. It is thought that these highly toxic compounds are not only involved in oral malodor but are also involved in the induction and/or progression of periodontal disease.

Halitosis is a condition that affects all individuals, either directly or indirectly, during some point in their lives and is a driving force motivating patients to seek dental care. Medically-compromised subjects, such as those afflicted with diabetes, lower respiratory tract infections, or trimethylaminuria, are particularly susceptible and may expel malodor from their oral cavity. This observation led to the screening of subjects with medically uncompromised health histories.

The screening was divided into three phases. Phase 1: A total of 50 subjects, aged 22 and older, with medically uncompromised health histories were analyzed. Medical histories were evaluated in the form of questionnaires. A halimeter was used to quantify VSCs exhaled during normal respiration. Three separate halimetric readings taken at 9:00 am, 1:00 pm, and 5:00 pm were recorded for each patient during the course of three days (data not shown). Phase 2: Cotton swabs were used to gather bacterial enzyme samples from three separate areas of the oral cavity (buccal/labial vestibule, posterior dorsum of the tongue, and the palate) for analysis (data not shown). Phase 3: The results were integrated into the development of clinical applications directed to combat halitosis.

It is widely accepted that the gram negative, anaerobic, pathogenic bacillus, Porphyromonas gingivalis, plays a major role in the production of endogenous VSCs. Porphyromonas gingivalis (P. gingivalis) is an oral pathogenic organism that produces volatile sulfur compounds (VSC's) in the buccal cavity and causes halitosis (Yoshimura et al. Infectious Immunology 68:6912-6916 2000; Nakano et al. Biochem. Biophys. Res. Commun. 292:964-968 2002; Nakano et al. Microbes and Infections 4:679-683 2002; Morita et al. Journal of Clinical Periodontology 28:813-819 2001). Few of the VSC's produced are hydrogen sulfide (H₂S), methanethiol and dimethyldisulfide. Certain sublingual microbiota via desulfhydrase activity produce H₂S and the deaminated product pyruvate, and ammonia from cysteine (Nakano et al. Biochem. Biophys. Res. Commun. 292:964-968 2002; Fukamachi et al. Biochem. Biophys. Res. Commun. 331:127-131 2005). P. gingivalis produces methylmercaptans through mgl (Yoshimura et al. Infectious Immunology 68:6912-6916 2000). Some of the VSC's have also been reported to initiate inflammatory reactions, damages to permeability of the oral mucosa, effects on collagen metabolism, and eventual periodontitis. The mgl gene has been cloned from P. gingivalis W83. The strain M1217 of P. gingivalis compared to W83 strain, contained similar levels of H₂S, however it had only ˜2.8% of methyl mercaptan (Nakano et al. Microbes and Infections 4:679-683 2002).

Considering that methionine-γ-lyase catalyzes the α,γ elimination of methionine to produce methanethiol (methyl mercaptan), α-ketobutyrate, and ammonia, it was implicated as a contributor to the pathogenesis of P. gingivalis. Thus, characterization of the enzyme was desirable.

Using specific oligonucleotide primers corresponding to methionine-γ-lyase (MEGL) (designed by DNA homology comparisons) and with genomic DNA of Porphyromonas gingivalis, cDNA corresponding to MEGL was amplified via PCR. The purified DNA was then cloned into bacterial overexpression vectors and the induced protein was purified and prepared in bulk for enzyme characterization (no data shown).

### Enzyme Assay:

Most of the assay protocols available regarding analysis of enzymatic products of MEGL involve use of impure proteins and measurement of methanethiol formed with indirect DTNB spectrophotometric assay. These assays are not particularly quantitative since they measure the volatile sulfur formed. Published assays involving measurement of non-volatile product α-ketobutyrate involve additional extractions, derivatizations, non-enzymatic processing, and spectrophotometry which contributes to inaccuracies and non-specificities.

MEGL Enzyme Isotope Assay Method:

This assay uses C¹⁴-labeled methionine and recombinant, purified, HIS-TAG cleaved and further purified MEGL protein. The MEGL enzyme forms very specific radiolabeled nonvolatile C¹⁴-labeled α-ketobutyrate. Further the products are separated by a silica gel-G thin layer chromatography (TLC) method using 75% acetonitrile and 25% 20 mM phosphate buffer (pH 7.0) solvent system. The substrate has an Rf value of 0.78 and the specific product has the Rf value of 0.82 as accurately determined by the exposure of TLC onto X-ray film and identifying the radioactive spots. Control experiments which lacked MEGL formed no C¹⁴-labeled α-ketobutyrate. This validates the enzyme procedure and the methods for analysis.

Further characterization of the enzyme was made using these protocols. MEGL exhibited pH optimum of 8.0 and an optimal temperature between 37-55° C. Various analogs (cystathionine, d-methionine, cysteine, O-succinyl-homoserine) exhibited less than 5% lyase activity. For 1-methionine, MEGL exhibited a km of 0.26 mM and a Vmax of 0.57 μmol/min/mg protein. The suicide inhibitor propargylglycine completely knocked out the MEGL activity. Therefore, low levels of this compound (propargylglycine) or its derivatives can be incorporated into mouth wash formulations to knock out the oral cavity-produced volatile sulfur compounds (VSCs) produced by pathogenic Porphyromonas gingivalis in order to prevent VSC production and the associated bad breath (halotosis) with minimal side effects. X-ray crystallography work can be performed to identify putative compounds that would have inhibitory activity.

### III. A Experimental: MEGL-2ABD in Cancer Summary of Experimental Procedures and Results

The experiments described herein test the concept that alterations in overall methionine metabolism will hamper cancer cell proliferation, differentiation, and renewal.

The experiments include the assay system that uses carboxyl labeled C¹⁴-methionine and homogeneous recombinant MEGL from P. gingivalis and the quantitative formation of the non-volatile C¹⁴-α-ketobutyrate in the overall reaction. Further, it is first demonstrated that MEGL/2ABD does not convert exogenous H³-L-2-aminobutyrate to form H³-α-ketobutyrate and ammonia confirming enzyme bound channeling and the overall enzymatic mechanism of deamination. Recombinant MEGL/2ABD, when expressed in methionine dependent HeLa cells, causes cell death and propargylglycine restores cells from death. BHK-21 (methionine independent) and HEK-AD293T cells transfected with MEGL/2ABD exhibited severe cell aggregation and propargylglycine inhibited MEGL/2ABD mediated cell aggregation.

Using genomic DNA of the oral pathogenic organism Porphyromonas gingivalis, cDNA corresponding to full length coding sequence of L-Methionine γ-Lyase/L-2-Aminobutyrate Deaminase (MEGL/2ABD) was amplified by PCR and cloned into a bacterial protein over-expression system. Genbank deposit (FJ875028) revealed an open reading frame of 1182 base pairs, 393 amino acids, <50% homology to most MEGL's and contained highly conserved motifs of β and γ carbon-sulfur lyases/aminotransferases. Over expressed purified protein exhibited a subunit Mw of ˜42.5 kDa. Purified protein was characterized by the novel isotope assay using 1-C¹⁴-methionine and the overall product C¹⁴-α-ketobutyrate was measured. The purified protein is a PLP-dependent bifunctional enzyme:L-methionine C^{γ}—S lyase/L-2-aminobutyrate deaminase that cleaves the γ-carbon sulfur bond of L-methionine into methanethiol and the deaminated overall product α-ketobutyrate and ammonia. The experiments demonstrate that exogenous ³H-L-2-aminobutyrate is deaminated into negligible ³H-α-ketobutyrate supporting efficient channeling of intermediate. Overall reaction of α-ketobutyrate formation exhibits a Km of 0.31 mM for methionine, Vmax of 0.67 μmol/min/mg and Kcat/Km of 1.3×10³ M⁻¹ sec⁻¹. MEGL/2ABD exhibited optimal activity above pH 8.0 and a temperature range between 37° C.-50° C. The relative activity of L-methionine in the presence of competing compounds were L-cystathionine (54.6%), L-cysteine (24.5%), D-methionine (43.6%), o-succinyl-homoserine (45.6%), selenomethionine (33.15%), sulfoxymethionine (46.71%) and the suicide inhibitor propargylglycine (0.26%). MEGL/2ABD cDNA was cloned into mammalian expression vector pEGFP-C3 and transfected into HeLa, HEK-AD293T and BHK-21 cells. HeLa showed significant cell death (30%) whereas HEK-AD293T and BHK-21 exhibited cell aggregation. Inclusion of propargylglycine in the culture medium restored viability in HeLa and prevented cell aggregation in HEK-AD293T and BHK-21.

### Detailed Experimental Procedures

Radionuclides (α carboxy-¹⁴ C Methionine and ³H-L-2-aminobutyrate) were purchased from Moravek. Oligonucleotides were custom made at the DNA core facilities of John's Hopkins University. Taq DNA polymerase and reagents for cloning were purchased from Invitrogen Inc. Thin-layer chromatography plates were from E. Merck (Darmstadt, Germany). Akta FPLC was from Amersham Biosciences, immobilized metal affinity column (IMAC) was obtained from (GE Healthcare, Inc.). The SDS-PAGE Mw standards (Unstained Precision Plus) were obtained from BioRad. All other compounds, solvents, and reagents were purchased from Sigma-Aldrich.

### Molecular Cloning and Bacterial Over Expression of Full Length MEGL/2ABD—

Using the gene sequence homology data from Genbank, PCR forward (5′-CAGACAGCTAGCATGCGTAGTGGCTTTG-3′) SEQ ID NO:3 and reverse (5′-GAACTCGAATTCTTAGATCAGGCTGTCCAGAC-3′) SEQ ID NO:4 primers were designed. With the genomic DNA of P. gingivalis (50 ng), forward and reverse primers, PCR was performed. The PCR products were then separated by 1% agarose gel electrophoresis using TAE buffer. A ˜1.2 kb product was then cloned into pGEM-T vector (Promega Inc.) between NheI and EcoRI sites. The cloned vector was transformed into JM109 of E. coli for further plasmid preparations. The insert was then excised from pGEM vector by overnight digestion with NheI and EcoRI, and ligated into a pET-28a-TEV over expression vector containing a 5′ hexa-His Tag and a TEV protease site upstream of the insert. Our sequence from P. gingivalis was deposited into Genbank (Accession number FJ875028) protein I.D. (AC094451.1) (DNA SEQ ID NO:1; amino acid SEQ ID NO:2) The novel vector, pMegL-ET, was transformed then into BL21 (DE3) (Invitrogen, Inc.) for over expression. The protein was then over expressed by the following method. Briefly a 2 liter culture of BL21 (DE3) containing the recombinant vector pMegL-ET was grown in Luria broth with kanamycin (40 μg/ml) at 37° C. until a mid log phase that showed A₆₀₀ of 0.4-0.6. Protein was then induced with IPTG at a final concentration of 1 mM and the bacterial cultures were incubated for 10-12 hours at 37° C. Cells were harvested at 5500 g and resuspended in a 40 mM Tris-HCl buffer (pH 7.3) that contained 5 mM imidazole and 250 mM NaCl (buffer A).

### Protein Purification

Resuspended frozen cells were thawed on ice, and treated with DNase I at 10 μg/ml and protease inhibitors (phenylmethylsulfonyl fluoride, 1 tablet of Complete Mini, EDTA Free, Protease Inhibitor (Roche) prior to lysis. Cells were lysed using a French Press at 20,000 PSI and the homogenate was clarified by centrifugation at 21,000 g at 4° C. (40 minutes). The supernatant was then purified through Ni⁺-NTA column and his-tag was cleaved by TEV protease according to standard procedures. 2 ml of His-Tag free proteins (10.2 mg) was applied on to a Superdex 200 (S-200) size exclusion column and eluted at a rate of 1 ml/min with [(storage buffer): 40 mM Tris-HCl (pH 7.3) that contained 1 mM DTT, 250 mM NaCl, 10% glycerol and 2 mM EDTA]. The peak fractions from S-200 column were then concentrated by Amicon (Mw cut off 10,000), which yielded 4.5-7.4 mg/ml protein. A ˜3 μg protein aliquot was tested for purity and subunit Mw, by 8-25% SDS-PAGE gel (PhastGel; GE Healthcare, Inc). Protein subunit Mw determination was performed using the molecular mass standards of Biorad (Unstained Precision Plus).

### Results: Molecular Cloning, Over Expression and Purification of MEGL/2ABD

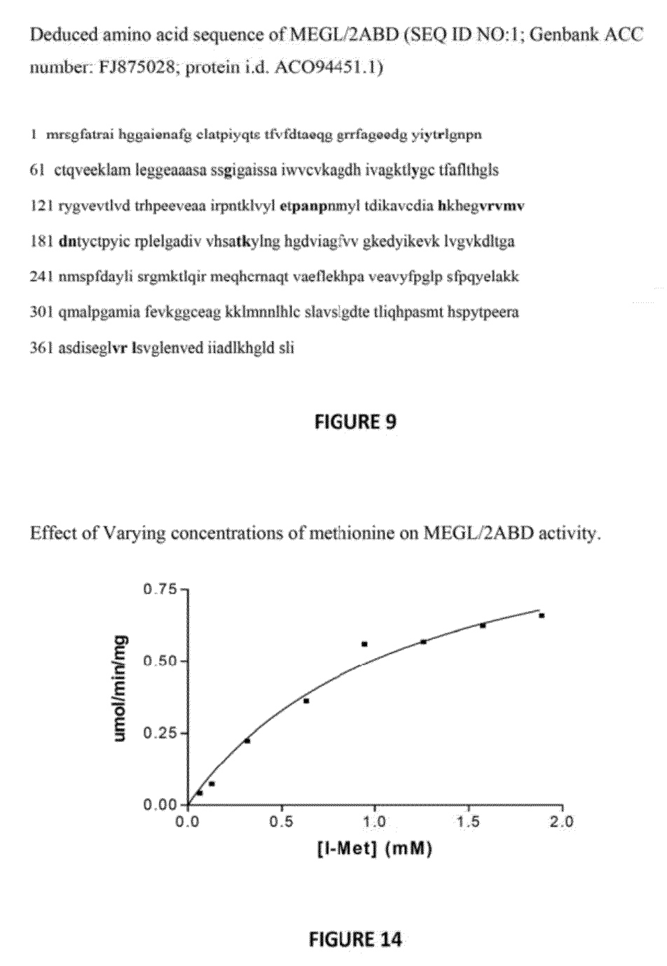

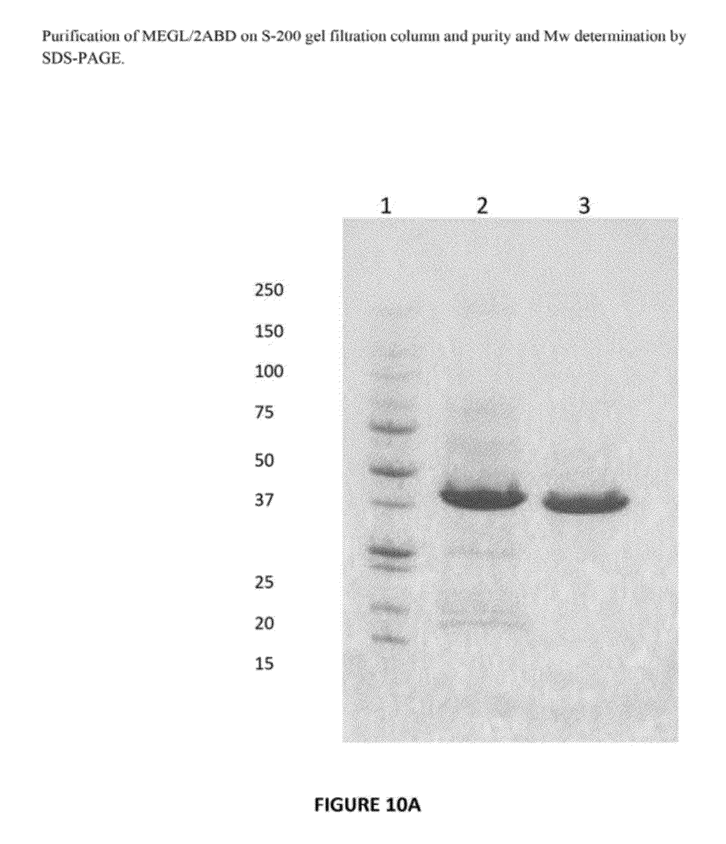

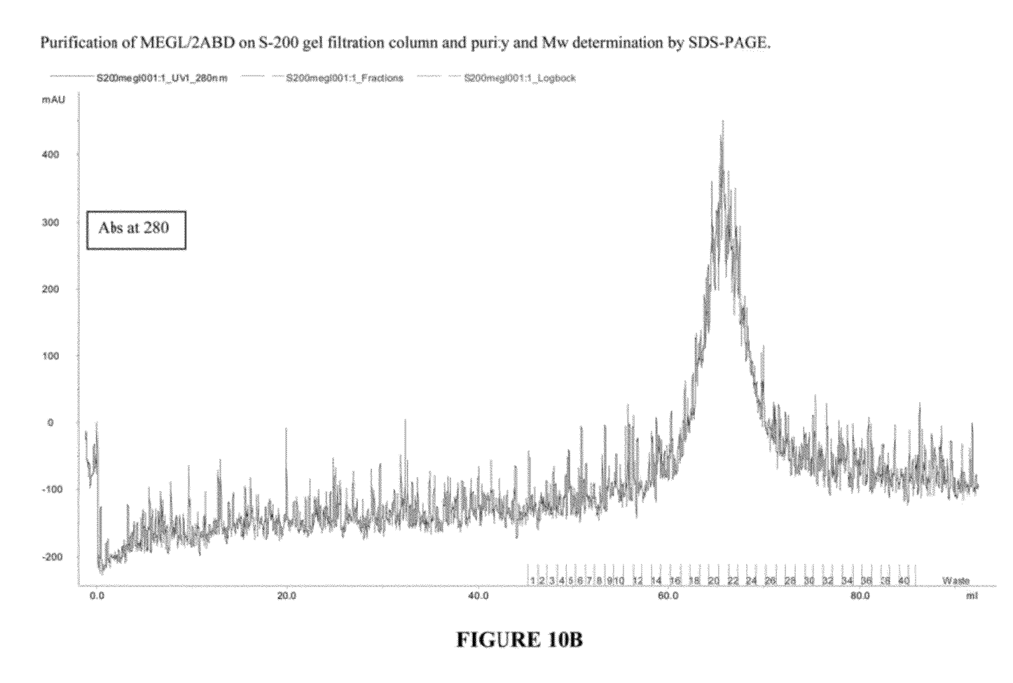

Using genomic DNA of the oral pathogenic organism p. gingivalis and homologous oligonucleotide primers, cDNA corresponding to full length coding sequence of MEGL was amplified by PCR and cloned into bacterial protein over expression system. The cDNA (SEQ ID NO:1) and protein (SEQ ID NO:2) sequences were deposited in GenBank (ACC number: FJ875028, protein I.D. AC094451.1). The deduced nucleotide sequence revealed an open reading frame of 1182 base pairs, amino acid residues of 393, <75% homology to all known MEGL, and <50% homology with most of the MEGL that has been reported. Our cDNA from P. gingivalis possessed highly conserved motifs of PLP binding and other putative active site residues found among β and γ carbon-sulfur lyases (FIG. 9). The cDNA sequence was compared for domain architecture with rest of the carbon-sulfur lyases (data not included). As described in detail in the methods the clarified bacterial cell lysate supernatant was purified through nickel column attached to FPLC. The eluted peak fractions contained mainly His-tag recombinant protein with minor impurities of lower molecular weight as evidenced by SDS-PAGE (lane 2, FIG. 10A). The active fractions from the nickel column were solvent exchanged as described in the methods. The His-tag moiety was removed using TEV protease, passed through Ni⁺ column, and the His-tag free recombinant protein was collected from the flow through and concentrated. Samples (10.2 mg) were further purified through superdex 200 gel filtration column (FIG. 10B). Comparison to a standard curve indicates a native, Mw of 160 kDa. Thus, P. gingivalis MEGL/2ABD forms a native homotetramer in solution under the given buffer conditions. Peak fractions from this column contained nearly homogeneous protein as evinced by SDS-PAGE (FIG. 10B).

FIGS. 9-10B: Molecular Cloning, Expression and Purification of MEGL/2ABD

(FIG. 9) cDNA (SEQ ID NO:1) and deduced amino acid (SEQ ID NO:2) sequences of MEGL/2ABD (Genbank ACC number: FJ875028; protein I.D. AC094451.1). Conserved PLP binding motif (Y52, R54, G83, T205), residues important for catalysis (Y108, E151, D181, N182, K206, R370), PLP schiff's base (K206) and aminotransferase motif (153PANP156, H171, 176VRVMV180, 369VRL371) were predicted based on the X-ray crystallographic structure resolved for β and γ carbon-sulfur lyases (Calvisi et al. International Journal of Cancer 121(11):2410-2420 2007; Ramani et al. Hepatology 51(3):986-995 2010; Hoffman, R. M. In Vitro 18:421-428 1982; Sun et al. Cancer Research 63:8377-8383 2003) (FIG. 10A) Gel filtration column chromatography of MEGL/2ABD on 124 ml Superdex-200 column. MEGL/2ABD elutes at 65 ml of solvent buffer. The column was run, ml/min and 1 ml fractions were collected. A tetramer was confirmed through comparison to a standard curve. (FIG. 10B) From the peak fractions of 5200 column chromatography, protein aliquots (3 μg) were tested for purity by SDS-PAGE (8-25%) gradient gel (phastGel; GE Healthcare, Inc). Lane 1. Mw ladder. Lanes 2. (protein with His-tag), lane 3. (protein without His-tag), The purified proteins from S-200 fractions analyzed are nearly homogeneous and possessed a subunit Mw of 42.5 kDa as judged by SDS-PAGE.

### Enzyme Assays Methionine γ-Lyase and Deaminase (MEGL/2ABD) Overall Activity Assay—

Typically the overall reaction of α-ketobutyrate formation was performed in a total volume of 24 μl. The reaction contained 4 μl of reaction buffer [150 mM Tris-HCl (pH 8.0)], 4 μl of 1 mM pyridoxal phosphate (PLP), 3 μl of H₂O, 6 μl of storage buffer, and 4 μl of diluted protein (0.4 μg), gently mixed and incubated at 37° C. for 5 minutes. The reaction was started by adding 1.93×10⁵ cpm of 1-¹⁴C-L-methionine (8814 cpm/nmol) and the contents were incubated at 37° C. for 15 minutes. The reaction was stopped by boiling for a minute, cooled on ice for 2 minutes and centrifuged at 3575 g for 7 minutes. A 2 μl aliquot was then chromatographed on a silica gel plate using 25% 20 mM phosphate buffer (pH 7.0) and 75% acetonitrile. Following chromatography, the TLC plates were dried, and exposed to x-ray film for 36-48 hours (Eastman-Kodak Co). The respective spots of substrate and products were cut out and radioactivity determined by liquid scintillation.

### Results: Enzyme Assay of MEGL/2ABD—

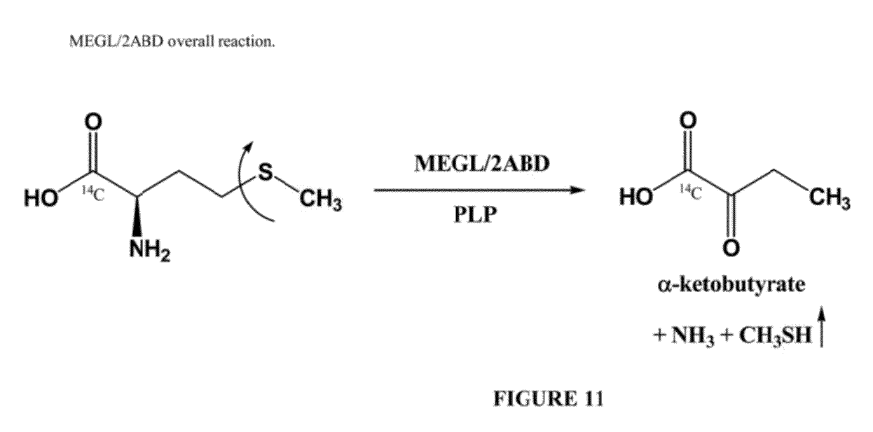

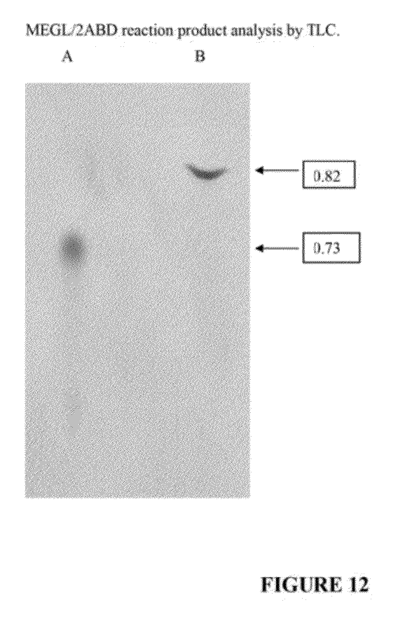

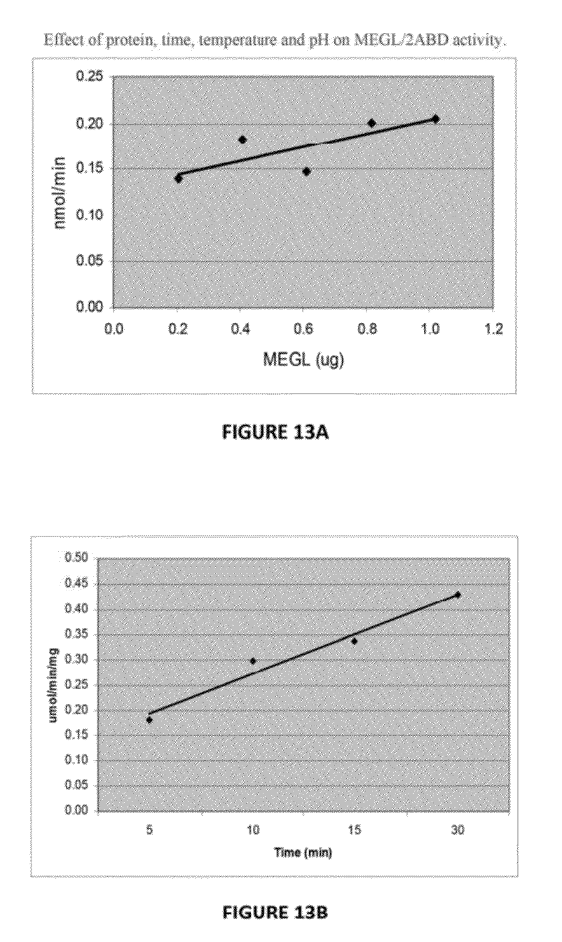

The purified protein was tested for MEGL/2ABD overall activity that monitored and analyzed the final product C¹⁴-α-ketobutyrate using the substrate 1-C¹⁴-L-methionine. The overall reaction is depicted in FIG. 11. As shown in FIG. 11, L-methionine is labeled at carboxyl carbon and after the cleavage and deamination reaction the product α-ketobutyrate would bear the label in its carboxyl carbon. The isotopic substrate 1-C¹⁴-L-methionine and the final product C¹⁴-α-ketobutyrate were separated by silica gel thin layer chromatography (TLC) using 75% acetonitrile and 25% 20 mM phosphate buffer. FIG. 12 shows the autoradiography of the TLC plates exposed on to film. Lane a, shows the quantitative conversion of the substrate into the final product C¹⁴-α-ketobutyrate (Rf 0.82) in the presence of all the components of reaction mixture plus the purified enzyme (>1 μg) incubated at 37° C. for 15 minutes. The reaction without the enzyme showed no conversion of substrate into any product as evidenced by the presence of 1-C¹⁴-L-methionine spot (Rf 0.78). This is the first report on this novel isotope assay system in which the non-volatile product (C¹⁴-α-ketobutyrate) is measured and quantitatively analyzed. Previous methods relied on the reaction of volatile methanethiol with DTNB and subsequent measurement of absorbance of the colored substance (thionitro benzene, TNB) at 410 nM. The characterization with DTNB assay was performed (data not shown) in which the cuvettes were overlaid with mineral oil in hope of preventing the escape of the volatile product methanethiol. However, with subjective smelling pungent sulfur odor was detected again confirming the loss of volatile product (methanethiol) underestimating the product formed relevant to DTNB assay. Nonetheless, preliminary spectrophotometric qualitative data helped to design the characterization of the enzyme with isotopic experiments. This isotope assay (Venkatachalam et. al., patent pending) is very accurate and measures the overall non-volatile product α-ketobutyrate. For routine isotope assays we used 0.4 μg of protein so that the substrate is not limiting during the course of 15 minutes of reaction time.

FIGS. 11-12: MEGL/2ABD Reaction.

(FIG. 11) MEGL/2ABD Overall Reaction. The substrate methionine was labeled (¹⁴C) at carboxyl carbon which upon cleavage and deamination would yield 1-¹⁴C-α-ketobutyrate. (FIG. 12) TLC separation of 2 μL aliquots of the reactions. Panel A: reaction that contained all the ingredients except MEGL/2ABD, shows substrate 1-C¹⁴-L-methionine, minor impurity below and no product. Panel B: reaction that contained enzyme shows only product C¹⁴-α-ketobutyrate.

### L-2-Aminobutyrate Deaminase (2-ABD)—

Assay-Typically the deamination reaction of ³H-2-aminobutyrate was performed in a total volume of 24 μl. The reaction contained 4 μl of reaction buffer [150 mM Tris-HCl (pH 8.0)], 4 μl of 1 mM pyridoxal phosphate (PLP), 6 μl of storage buffer, and 4 μl of diluted protein (0.402-4.02 μg), gently mixed and incubated at 37° C. for 5 minutes. The reaction was started by adding 6 μCi of ³H-2-aminobutyrate (3.108×10⁶ cpm/nmol) and the contents were incubated at 37° C. for 15 minutes. Further processing, chromatography, and analysis were done in the same manner as the overall activity assay.

### Cloning and Expression of MEGL in Mammalian Cells and Confocal Microscopy—

The recombinant vector pMEGL-ET containing MEGL insert and pEGFP-C3 mammalian expression vector (Promega, Inc.) were digested with EcoR I and Bgl II restriction enzymes. The digested products were separated on 1% agarose to verify digestion and the MEGL insert was ligated into pEGFP-C3 vector. The ligated vector was then transformed into DH5α bacteria and the recombinant colonies were selected by kanamycin resistance. The plasmid from the recombinant colonies, were further tested for the insert by PCR using MEGL/2ABD specific primers. The recombinant bacteria, was then grown in bulk, plasmid isolated by maxi/mini preps using columns (Qiagen, Inc.). Purified plasmid was then used for mammalian transfection. HeLa, HEK-AD293T and BHK-21 cells (ATCC, Rockville, Md.) were grown in 75-cm² flasks in Dulbecco's modified Eagle's medium, supplemented with 10% fetal bovine serum 100 units/ml penicillin and 100 μg/ml streptomycin (Invitrogen Inc), in an atmosphere of 5% CO₂, 95% air at 37° C. For confocal microscopy, cells were plated on cover slips in 35 mm² dishes. When cells reached 50-75% confluency they were placed in serum and antibiotic-free medium and co-transfected with plasmid DNA containing GFP and with or without MEGL/2ABD insert. Transfections were achieved with LipofectAMINE® 2000 (Invitrogen, Inc.) according to the manufacturer's instructions. Transfections of HEK-AD293T cells were performed using standard calcium phosphate methods. After incubation of cells for 4-18 hours, cells were washed with phosphate-buffered saline, fixed with 4% formaldehyde for 8 minutes, permeabilized using 0.2% Triton X-100 (Sigma-Aldrich) and mounted on glass slides using the ProLong Antifade Kit (Molecular Probes) for staining with DAPI using standard protocols. Samples were examined on a Zeiss LSM510 confocal microscope.

### Cell Viability Assay—

The effect of MEGL on cell viability was determined using 3-[4,5-dimethylthiazol-2-yl]-2,5-diphenyl-tetrazolium bromide (MTT, Sigma-Aldrich). For MTT assay, cells were suspended at a concentration of ˜4×10⁴ cells (180 μl/well). 20 μl of MTT solution (5 mg/ml) was added for each well and incubate at 37° C. and 5% CO₂ for about 2-5 hours for purple color development. Formazan precipitates were dissolved in DMSO for 30 minutes at 37° C., and the absorbance was measured at 570 nM.

### Results: Characterization of MEGL/2ABD—

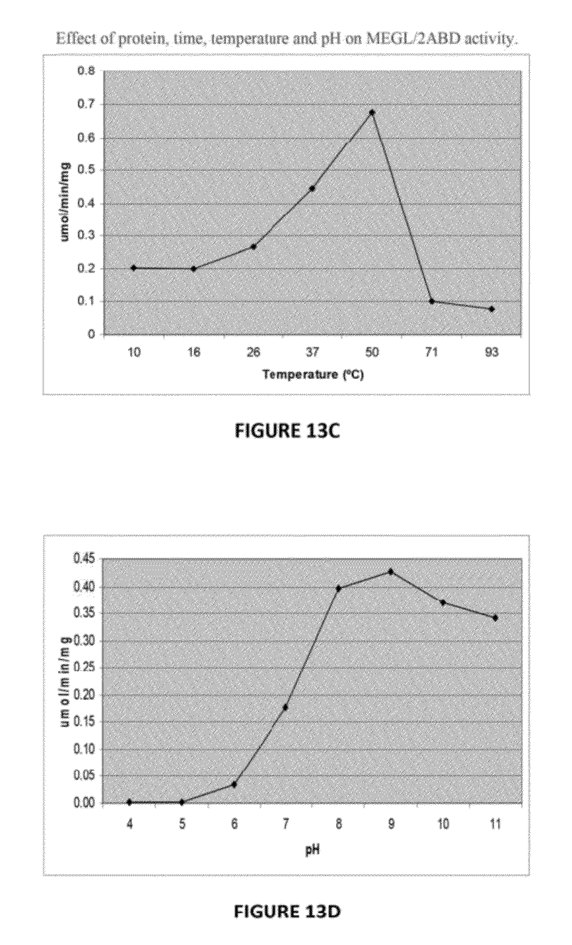

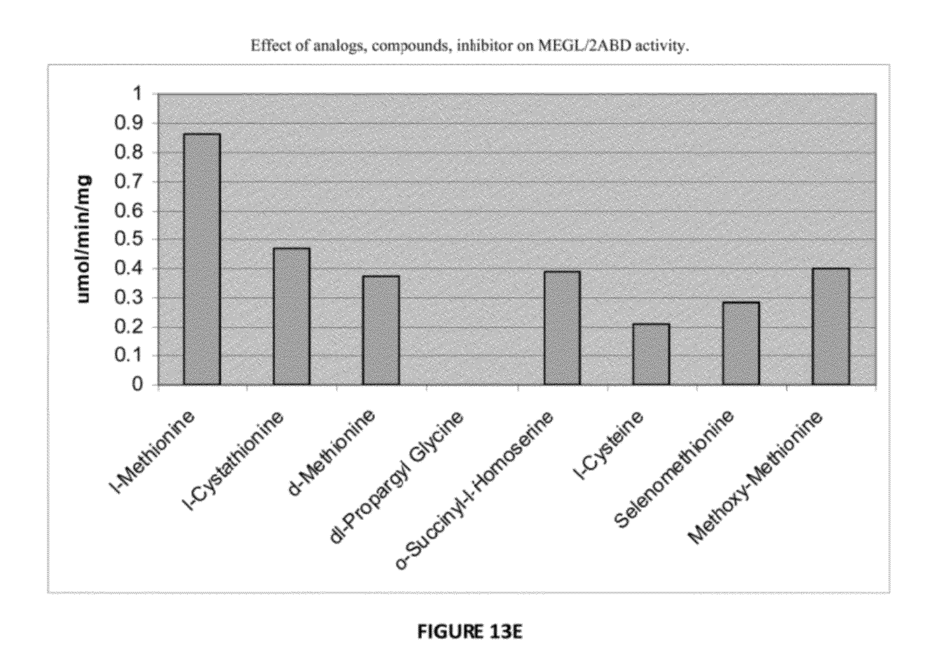

The activity was measured using different amounts of protein ranging from 0.1 to 1 μg and the reaction was performed for 5-30 minutes. The reaction was linear with both parameters (FIGS. 13A-E). For routine assays we chose a protein concentration of 0.4 μg and a reaction time of 15 minutes and a temperature of 37° C., which ensured substrate availability. The enzyme activity was very low at pH below 6.0 and showed an optimal activity at pH 8.0 and higher as shown in FIGS. 13C and 13D. The MEGL/2ABD exhibited optimal activity between temperature ranges of 37-55° C. The relative percent activity of the various compounds tested was similar to the isotope assay as judged by indirect DTNB qualitative assay (data not reported). The relative MEGL/2ABD activity in the presence of competing substrates were L-cystathionine (54.6%), D-methionine (43.6%), O-succinyl L-homoserine (45.6%), L-cysteine (25.5%), seleno-methionine (33.2%), methionine-sulfoxide (46.7%). The activity was dramatically reduced to 0.26% in the presence of the suicide inhibitor propargyl glycine (FIG. 13E).

FIGS. 13A-E. MEGL/2ABD Characterization.

(FIG. 13a) and (FIG. 13b) Effect of protein and time on MEGL/2ABD activity. Reaction at each protein concentration was performed for 15 minutes at pH 8.0 according to the standard assay procedures described. Effect of time was tested using 0.4 μg of protein. (FIG. 13C) Effect of temperature on MEGL/2ABD activity. Reaction at each temperature was performed for 15 minutes at pH 8.0 with 0.4 μg of protein according to the standard assay procedures described. (FIG. 13D) Effect of pH on the MEGL/2ABD activity. Reactions were performed for 15 minutes at 37° C., using reaction buffers of various pHs that contained ingredients used in standard assays described. [The following buffers were used for various pHs: citrate reaction buffer (pH 4.0, 5.0, and 6.0); phosphate buffer (pH 6.0 and 7.0); Tris-Hcl (pH 8.0 and 9.0), and glycine (pH 9.0, 10.0, and 11.0)]. (FIG. 13E) Effect of analogs/inhibitors on MEGL/2ABD activity and it shows the relative activity of MEGL/2ABD in the presence of various compounds.

### Kinetic Analysis—

Purified enzyme preparations that contained high enzymatic activity were used for determining the kinetic parameter Km for L-methionine. The overall reaction of α-ketobutyrate formation exhibited a Km of 0.31 mM for methionine, Vmax of 0.67 μmol/min/mg and a Kcat/Km of 1.3×10 ³ M⁻¹ Sec⁻¹ (FIG. 14).

### FIG. 14. MEGL/2ABD Kinetic Analysis, Against L-Methionine Substrate.

Purified enzyme was assayed against varying concentration of L-methionine, as described. Data points represent an average of two independent experiments. Km and Vmax values were determined from GraphPad Prism (GraphPad Software, Inc.).

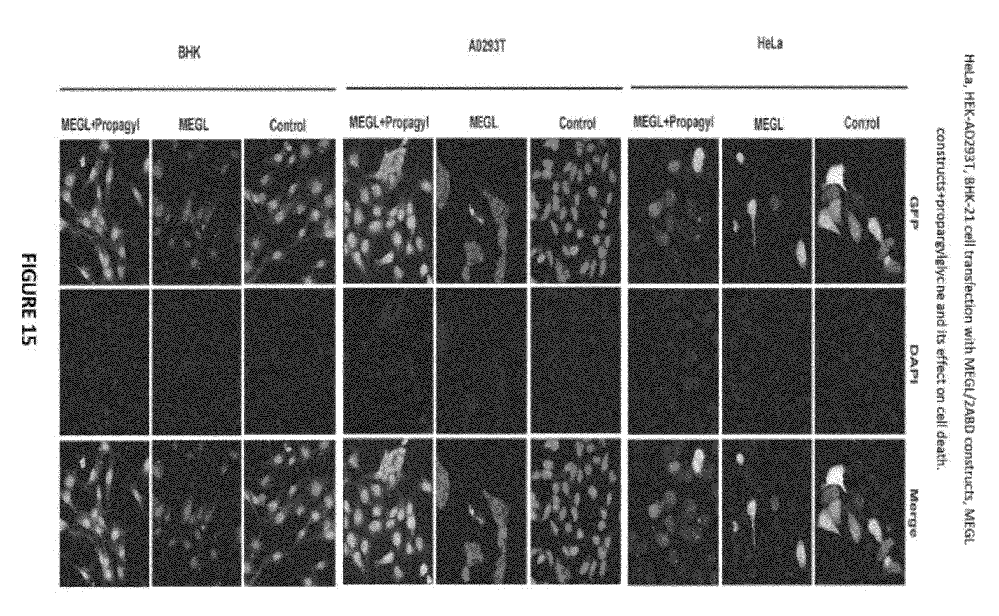

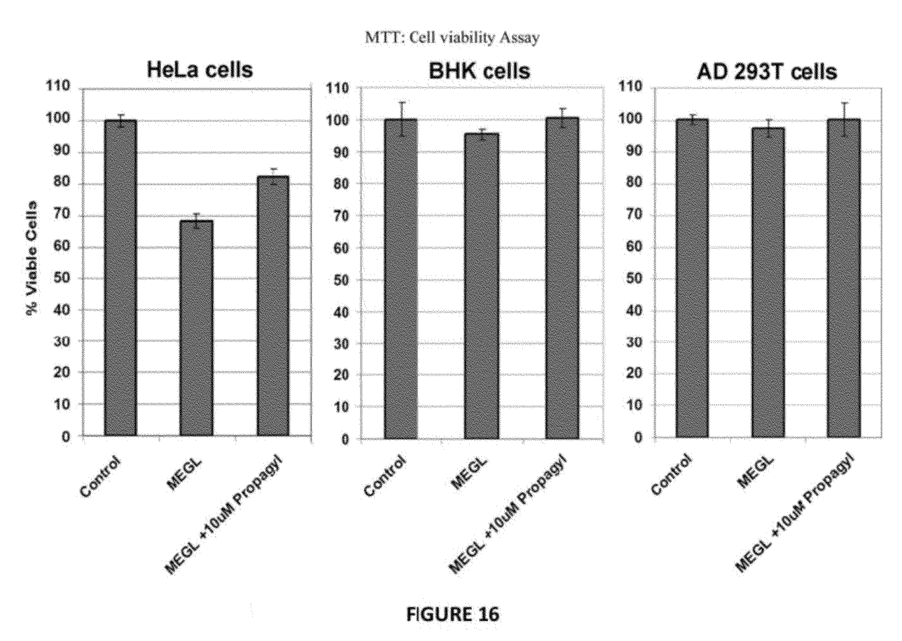

Mammalian Cell Expression of MEGL/2ABD and Cell Viability—The MEGL/2ABD cDNA was cloned into mammalian pEGFP-C3 vector and transfected into HeLa, HEK-AD293T and BHK-21 cells. Significant transfection was seen within 4 hours as evidenced by GFP expression (data not shown). Cell counts were similar in both control (GFP alone) and experimental (GFP+MEGL/2ABD), 4 hours after transfection as evidenced by DAPI staining and MTT assays (data not shown). However HeLa cells, after 18 hours of transfection, GFP+MEGL/2ABD construct showed significant cell death as evidenced by confocal images of GFP and DAPI staining Proparaglyglycine is a potent inhibitor of MEGL/2ABD as we have shown with the purified enzyme kinetics. Treatment of cells with 10 μM propargyl glycine 2 hours after transfection in the culture medium restored nearly all cells from death (FIG. 15). Cell viability measured by MTT assay showed 32 (+/−2.2) % of Hela cell death (FIG. 16). However, HEK-AD293T and BHK-21 cells transfected with MEGL constructs showed very prominent cell aggregation. Treatment of cells with 10 μM propargylglycine 2 hours after transfection in the culture medium inhibited the cell aggregation that is prominently evident as shown by the confocal images (FIG. 15). Interestingly, cell viability measured by MTT assay showed no significant cell death of HEK-AD293T and BHK-21 (FIG. 16).

### FIG. 15. Confocal Microscopy Picture of Fixed, PBS Washed Cells.

After 18 hour incubations cells were washed with phosphate-buffered saline, fixed, permeabilized and mounted on glass slides using the ProLong Antifade Kit (Molecular Probes) with DAPI staining as previously described by Enniga et al. 2006. Samples were examined on a Zeiss LSM510 confocal microscope.

### FIG. 16. MTT Assay:

Viability of cells transfected with MEGL construct. Three different cell lines (Hela, BHK-21, and HEK-AD293T) were plated in 96-well plates and transfected with either vector expressing green fluorescent protein, GFP (pEGFP-C3), or MEGL and GFP (pEGFP-C3-MEGL). After 18 hours of transfection, cells were treated with MTT and viability was determined by measuring the MTT absorbance at λ570 nm. Control absorbance was arbitrarily determined as 100% viability. HeLa cells show an average of 32 (+/−2.2) % decrease in cell viability with 18 hour MEGL expression. Under similar conditions, BHK-21 and HEK-AD293T cells show an average of 4.6 (+/−1.6) %, and 2.7 (+/−2.7) % decrease in cell viability, respectively.

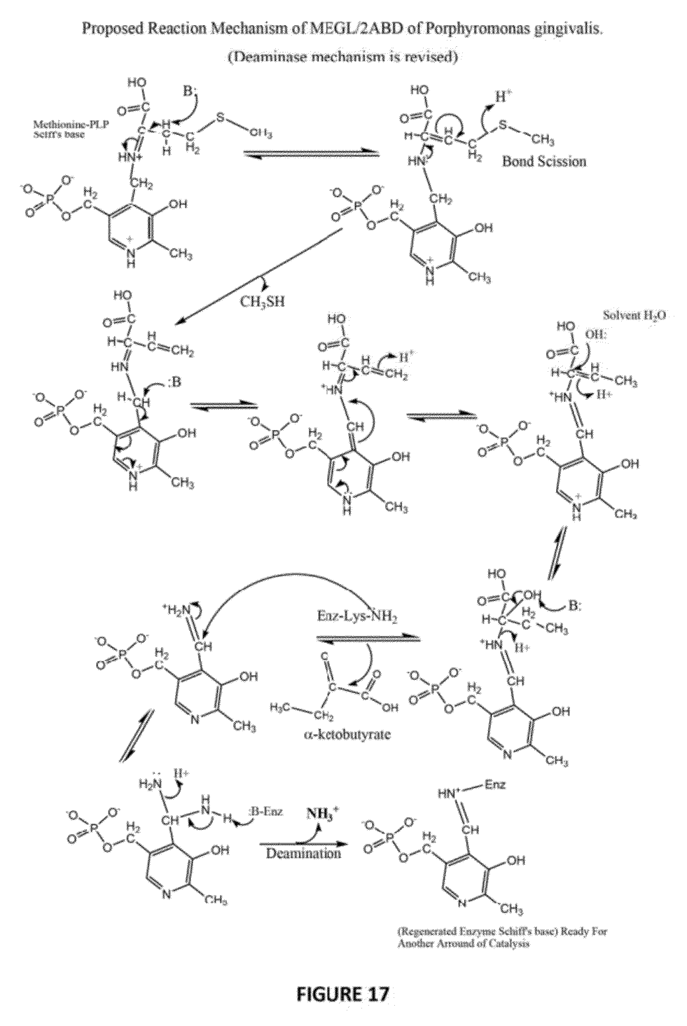

### FIG. 17. Proposed Reaction Mechanism of MEGL/2ABD.

The γ carbon-sulfur lyase part of the reaction mechanism in forming the methanethiol and enzyme bound intermediate is very similar to the one that is described for Clostridium sporogenes (Hadjipanayis et al. Trends in Molecular Medicine 11:519-530 2009). However, the deaminase part of the reaction mechanism has been revised (Venkatachalam et. al. unpublished). In this mechanism it is proposed that the enzyme bound diamine intermediate formed before ammonia release. This was supported by the overall reaction occurring in the range of enzyme catalytic rates.

### Experimental Conclusions

The novel isotope labeled at the carboxyl carbon and the overall reaction measures the formation of C¹⁴-α-ketobutyrate by silica gel TLC separation. The assay is very specific, i.e. without the enzyme there were no product spots corresponding to α-ketobutyrate. However, in the presence of >4.0 μg of purified protein, in 15 minutes there was quantitative formation of product and no intermediate 2-aminobutyrate, as shown by autoradiography and liquid scintillation. For routine assays, the reaction was run for 15 minutes at 37° C. confirming the substrate availability during reaction. Without the enzyme there was no conversion into products proving overall rate of reaction is enzymatic. The isotopic substrate from Moravek (claimed 99.4% radiochemical purity) contained impurity that separated well in our TLC system (Rf 0.4) and in the presence of enzyme there was only one product spot. Therefore, it is possible that the impurity could be a sulfur amino acid contaminant such as L-homocysteine that might have been used for methionine preparation by the manufacturer.

The cDNA from P. gingivalis, had <50% homology to most of the MEGL's reported and <70% homology with all of the MEGL's that have been reported thus far. The deduced sequence (Genbank ACC number: FJ875028; SEQ ID NO;1) revealed an open reading frame of 1182 base pairs, amino acid residues of 393 (protein I.D.: AC094451.1; SEQ ID NO:2), with highly conserved motifs of PLP and other putative active site residues found among many β and γ carbon-sulfur lyases (Calvisi et al. Int. J. Cancer 121(11):2410-2420; Ramani et al. Hepatology 51(3):986-995 2010; Hynes, R. O. Science 326(5957):1216-1219 2009).

From the protein primary sequence comparisons and X-ray crystallographic information of PLP dependent enzymes (Clausen et al. Journal of Molecular Biology 262:202-224 1996) and MEGL from various organisms (Sridhar et al. Acta. Cryst. D56: 1665-1667 2000; Nikulin et al. Acta. Cryst. D64:211-218; Mamaeva et al. Acta Crystallogr Sect F Struct Biol Cryst Commun. 61 (Part 6):546-549 2005; Motoshima et al. Journal of Biochemistry 128:349-354 2000) an overall tri domain structure is proposed (N-terminal substrate entry, large PLP binding and carboxy terminal domain) for the monomer of our P. gingivalis MEGL/2ABD. It is also proposed that the dimer and tetramer catalytic active site interface formation of MEGL/2ABD will be very similar to CBS and CBL's and other MEGL's (X-ray crystallography in progress).

The highly purified protein exhibited a Km of 0.31 mM for methionine and a Vmax of 0.67 mmol/min/mg and Kcat/Km of 1.3×10 ³ M⁻¹ Sec⁻¹. The reported Km value of MEGL/2ABD is well within the reasonable physiological concentration of methionine (−0.6-0.8 mM) in prokaryotic cell that was measured by NMR and direct amino acid analysis (Park et al. Journal of Clinical Investigation 120(2):636-644 2010). Numerous other reports have shown Km values (ranging between 0.61-3.58 mM and 6-90 mM) that are very high perhaps due to limitations of the assays that were employed, for instance a) DTNB assay that would measure the volatile sulfur compounds formed, or b) the volatile sulfur, could air oxidize and form disulfides, that are not reactive with DTNB which again would underestimate the product formed. Another limiting assay involves the measurement of α-ketobutyrate formed by derivatizing it and subsequently measuring the quantity of it by absorption at 312 nM by spectrophotometry. This aside, higher Km values reported [e.g. for Entamoeba histolytica (3.58 mM), Trichomonas vaginalis (4.3 mM)] (Fabian et al. Cytometry Part A 75A:67-74 2009; Park et al. Journal of Clinical Investigation 120(2):636-644 2010; Okuno et al. Oncology Reports 2:485-492 2010; Mueller et al. Front Bioscience (Elite Edition) 2:602-613 2010; Telford, W. G. Current Protocols in Cytometery Chapter 9: Unit 9.30 2010) were due to one of the two isoforms that has less specificity towards L-methionine and more specificity towards other sulfur amino acids.

The purified protein of P. gingivalis exhibited maximal activity between temperature ranges of 37-55° C. Although this is a paradox of how it would have any physiological relevance, this phenomenon can be of use while handling the protein for various in vitro manipulations such as incorporating protein into nanoparticles for solid tumor targeted therapeutics.

Methionine can be resynthesized from homocysteine by methionine synthase which requires the coenzyme vitamin B₁₂ derivative adenosylcobalamin (Hoffman et al. Proceedings of the National Academy of Science USA 73:1523-1527 1976). The importance of methionine in human cell physiology is thoroughly described (Hoffman et al. Proceedings of the National Academy of Science USA 73:1523-1527 1976). The role of methionine in cancer cells had been studied in depth (Hori et al. Cancer Research 56:2116-2122 1996; Takakura et al. Cancer Research 66:2807-2814 2006; Kim et al. Journal of Cellular Physiology 212:386-400 2007). Methionine dependent cancer cells (Kim et al. Journal of Cellular Physiology 212:386-400 2007) (for e.g. 293T, and HeLa) apparently have defective methionine resynthesis from homocysteine, methyltetrahydrofolate and cobalamin, catalyzed by endogenous tumor specific isoform of methionine synthase complex (E.C. 2.1.1.13; identification number from “The Comprehensive Enzyme Information System” database). The effect of MEGL/2ABD on HeLa cells was tested and shows that the cells undergo significant apoptosis. However, HEK-AD293T and BHK-21 a methionine independent cells when transfected with MEGL/2ABD construct showed no cell death even after 18 hours, however showed significant cell aggregation as evidenced by the confocal images. This suggests that in these two cell types, the endogenous methionine synthase perhaps forms methionine from homocysteine. Further experiments are under way to study the detailed cell physiology of these cell lines in terms of methionine pool, methylcobalamin (vitamin B₁₂ derivative) and N₅-methyl-Tetrahydrofolate (N₅-methyl-THF) dependent methionine synthase activity through re-synthesis. Effects of folate and cobalamin will be studied in the presence of MEGL in these cell types. MEGL from various sources had been used for cancer therapeutics (Weissbach, H. Journal of Biological Chemistry 35:23497-23504 2008; Foster et al. Nature 201:39-42 1964; Guest et al. Biochemistry Journal 92:497-504 1964). Therapeutically this is an excellent strategy with P. gingivalis MEGL/2ABD (Venkatachalam et. al. patent pending).

The enzyme activity was very low at pH <6.0 and had optimal activity at pH >8.0. Assuming that the pKa of the active site lysine is basic, one would expect that the enzyme bound PLP would be able to form a Schiff's base and mechanistically maximize the chance of formation of the overall product α-ketobutyrate.

Previous IUBMB nomenclature description of the reaction mechanism had shown that the 2-amino-enebutyrate would be deaminated by non-enzymatic mechanisms into 2-oxobutyrate and ammonia. Based on the results, it is proposed, that the deaminase part of the MEGL/2ABD perhaps forms an enzyme bound diamine intermediate before releasing ammonia (FIG. 17). In this mechanism the bound intermediate would be deaminated to form ammonia and α-ketobutyrate, simultaneously regenerating the PLP-bound enzyme Schiff's base for next round of catalysis. Thus, there is a clear involvement of enzyme through the entire reaction mechanism to form the overall product α-ketobutyrate. Since the rate of catalysis Kcat/Km of 1.3×10 ³ M⁻¹ Sec⁻¹ is in the mid range of reported enzyme rates, it is predicted that the overall reaction is enzymatic as opposed to the previous nonenzymatic mechanism of deamination of 2-amino-enebutyrate. In the assays, the overall α-ketobutyrate formation at temperatures 72-93° C. were very minimal, disproving the non-enzymatic mechanism of ammonia release from 2-aminobutyrate to form α-ketobutyrate. Independently, the exogenous ³H-2-aminobutyrate along with MEGL/2ABD (4 μg) did not form any α-ketobutyrate. Whereas, with the starting substrate L-methionine and less amounts of protein (1 μg), the reaction was quantitative in forming α-ketobutyrate. This could mean that at the active site, there is direct channeling of the enzyme bound intermediate to form α-ketobutyrate without any accumulation of the intermediate. Thus, a new name has been coined for our bifunctional enzyme as L-methionine ^{γ}C—S-lyase/L-2-aminobutyrate deaminase (MEGL/2ABD).

Further kinetic experiments are underway to establish the diamine intermediate. It is further reported that recombinant MEGL/2ABD when expressed in mammalian cells causes cell death in methionine dependent HeLa cells. The effect of MEGL/2ABD in dwindling intracellular methionine and in addition the effect of intracellular pH changes due to ammonia release in methionine dependent (BHK-21) and methionine independent (BHK-21A8) cell lines is also being studied. Incorporation of MEGL/2ABD into targeted drug delivery systems such as nanoparticle and retrovirus vectors for cancer therapeutics of methionine dependent tumors and perhaps metastatic cancers is intended for the future.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The proteins, peptides, nucleotides, methods, procedures, and techniques described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

1. A method for quantitatively determining enzyme activity of a methionine gamma lyase-2 aminobutyrate deaminase (MEGL-2ABD) protein, the method comprising the steps of:
providing a reaction mixture including a known quantity of purified MEGL-2ABD protein;
beginning a reaction by adding a known quantity of a radiolabeled substrate for the purified MEGL-2ABD protein to the reaction mixture, wherein the radiolabeled substrate is 1-¹⁴C-L-methionine;
heating the reaction mixture for a period of time;
stopping the reaction;
separating a product and remaining substrate from the reaction mixture; and
determining an amount of product formed from the reaction, whereby the amount of product determined quantifies the enzyme activity of the purified MEGL-2ABD protein.

2. The method according to claim 1, wherein the step of providing includes providing a reaction mixture including known quantities of reaction buffer, pyridoxal phosphate, water, and storage buffer.

3. (canceled)

4. The method according to claim 1, wherein the step of heating the reaction mixture for a period of time includes stirring the reaction mixture and heating the reaction mixture for five minutes.

5. The method according to claim 1, wherein the step of stopping the reaction includes boiling the reaction mixture for one minute, cooling the reaction mixture on ice for two minutes, and centrifuging the reaction mixture for seven minutes.

6. The method according to claim 1, wherein the step of separating a product and remaining substrate from the reaction mixture includes chromatography using a gel plate.

7. The method according to claim 6, wherein the step of determining an amount of product formed from the reaction includes removing separated product and substrate from the gel and measuring radioactivity of the product and substrate using liquid scintillation.

8-13. (canceled)

14. A method for altering methionine metabolism in a cell comprising:
providing a composition including a methionine gamma lyase-2 aminobutyrate deaminase (MEGL-2ABD) protein; and
administering the composition to the cell.

15. The method according to claim 14, wherein the MEGL-2ABD protein comprises SEQ ID NO:2.

16. The method according to claim 14, wherein the cell is a cancer cell.

17. The method according to claim 16, wherein the cancer cell is selected from the group consisting of cancer stem cells, differentiated cancer cells, and proliferating cancer cells.

18-21. (canceled)

22. A method for ameliorating cancer in a subject in need thereof comprising:
providing a composition including a methionine gamma lyase-2 aminobutyrate deaminase (MEGL-2ABD) protein and a pharmaceutically-acceptable carrier; and
administering the composition to the subject.

23. The method according to claim 22, wherein the MEGL-2ABD protein comprises SEQ ID NO:2.

24. The method according to claim 22, wherein the pharmaceutically-acceptable carrier is a nanoparticle or a retroviral vector.

25-37. (canceled)

38. The method according to claim 1, wherein the step of separating a product and remaining substrate from the reaction mixture includes separating C¹⁴ radiolabeled alpha ketobutyrate and 1-¹⁴ C-L-methionine.

39. The method according to claim 14, wherein altering methionine in a cell includes reducing intracellular methionine in the cell.

40. The method according to claim 14, wherein altering methionine in a cell includes increasing intracellular toxicity in the cell.

41. The method according to claim 22, wherein ameliorating cancer in a subject in need thereof includes inducing death of cancer cells.

42. The method according to claim 41, wherein the cancer cells are selected from the group consisting of cancer stem cells, differentiated cancer cells, and proliferating cancer cells.


SEQUENCE LISTING (4 sequences)

SEQ ID NO: 1 - DNA, 1182 bp, Porphyromonas gingivalis
atgcgtagtggctttgccacacgtgccatccatggaggcgctatcgagaacgccttcggctgcttagccactcccatttaccaaacatcgactttcgtttttgacactgccgaacagggaggccgccgctttgccggagaggaagacggatacatctatacccgtctgggcaaccccaactgcacccaagtggaagagaaactggccatgctcgaaggcggagaagccgccgcatcggcctcatccggtatcggagccatcagctctgccatctgggtatgcgtgaaggccggcgaccatatcgtagccggcaagacgctctacggctgcaccttcgccttcctcacccacggactgagccgctacggtgtggaagtcaccctcgtggatacccgccatccggaagaggtggaggccgccattcgcccgaatacgaagctcgtctatctggagactccggccaaccccaatatgtacctgaccgacatcaaggcagtctgcgacatcgctcataagcacgaaggcgtacgcgtcatggtggacaatacctactgcacgccctatatctgccgtccgctggagctgggtgccgacatcgtggtacacagcgcgaccaagtacctgaacggacatggcgacgtcatcgccggattcgtcgtaggtaaagaggactacatcaaggaggtgaagctcgtcggcgtcaaggacctcacgggggccaatatgagtccgttcgatgcttatctgatcagccgcggcatgaagacgctgcagatacgtatggagcagcactgccgcaatgctcagaccgtagccgaatttctcgaaaagcatccggccgtagaagcagtttatttccccggacttccgagcttcccccaatacgaattggccaagaagcagatggcactgcccggagccatgatcgccttcgaagtgaagggcggttgcgaagccggtaagaagctgatgaacaacctgcacctctgctccctcgccgtgagcttgggcgatacggaaaccctcatccagcatccggccagcatgacccactcgccctacacacccgaagagcgtgctgccagcgacatatccgaaggactggtacgcctgtccgtgggtctggagaacgtggaggacatcatcgccgacctcaagcacggtctggacagcctgatctaa

SEQ ID NO: 2 - protein, 393 aa, Porphyromonas gingivalis
MRSGFATRAIHGGAIENAFGCLATPIYQTSTFVFDTAEQGGRRFAGEEDGYIYTRLGNPNCTQVEEKLAMLEGGEAAASASSGIGAISSAIWVCVKAGDHIVAGKTLYGCTFAFLTHGLSRYGVEVTLVDTRHPEEVEAAIRPNTKLVYLETPANPNMYLTDIKAVCDIAHKHEGVRVMVDNTYCTPYICRPLELGADIVVHSATKYLNGHGDVIAGFVVGKEDYIKEVKLVGVKDLTGANMSPFDAYLISRGMKTLQIRMEQHCRNAQTVAEFLEKHPAVEAVYFPGLPSFPQYELAKKQMALPGAMIAFEVKGGCEAGKKLMNNLHLCSLAVSLGDTETLIQHPASMTHSPYTPEERAASDISEGLVRLSVGLENVEDIIADLKHGLDSLI

SEQ ID NO: 3 - DNA, 28 bp, Artificial Sequence
  forward primer for expression of SEQ ID NO2 using PCR
cagacagctagcatgcgtagtggctttg

SEQ ID NO: 4 - DNA, 32 bp, Artificial Sequence
  reverse primer for expression of SEQ ID NO2 using PCR
gaactcgaattcttagatcaggctgtccagac